(19) United States
(12) Patent Application Publication
(10) Pub. No.: US 2013/0296807 A1
(43) Pub. Date: Nov. 7, 2013

(54) DRUG RECONSTITUTION AND DELIVERY DEVICE

(71) Applicants: Richard Lintern, Cambridge (GB); Edward Murray, Cambridge (GB); Oliver Shergold, Bolligen (CH); Oliver Boris Stauch, Freiburg (DE)

(72) Inventors: Richard Lintern, Cambridge (GB); Edward Murray, Cambridge (GB); Oliver Shergold, Bolligen (CH); Oliver Boris Stauch, Freiburg (DE)

(73) Assignee: F. HOFFMANN-LA ROCHE AG, Basel (CH)

(21) Appl. No.: 13/813,734

(22) PCT Filed: Aug. 10, 2010
(86) PCT No.: PCT/EP10/61634

§ 371 Date: Jun. 25, 2013

(51) Int. Cl. A61J 1/20; A61M 5/158

(52) U.S. Cl. CPC A61J 1/20; A61M 5/158
USPC 604/272; 604/416; 604/414; 604/404

(57) ABSTRACT

A device for automatically delivering a drug to a user and method thereof are disclosed. The device (10, 20, 70, 110) provides a connector to which a vial (12, 50, 60, 100, 130, 202) containing the drug is removably connected and situated thereto in a substantially inverted position. When commanded by the user, the device (10, 20, 70, 110) starts a reconstitution process to automatically create a reconstituted drug, and provides an indication that the reconstitution process is complete such that the vial (12, 50, 60, 100, 130, 202) may be removed from the connector (22, 72,112). After disposing the device (10, 20, 70, 110) on the user, the device (10, 20, 70, 110) can automatically deliver the reconstituted drug to the user when further commanded by the user.

### TECHNICAL FIELD

The present disclosure generally relates to a drug delivery device for a drug, or multiple drugs that require diluting and/or mixing and, more specifically, to a device which automatically reconstitutes a drug and automatically delivers the reconstituted drug to the user.

### BACKGROUND

As background, certain drugs may not be stable in a liquid solution for long-term storage, or must be diluted from a more stable concentrated liquid form prior to administration (collectively “reconstitution”). For example, in some instances drug solutions are lyophilized into a powdered form using a freeze-dry or other similar process. A lyophilized drug may then be suitable for long-term storage and may be converted back into a liquid form when it is ready to be used. Furthermore, some combinations of drugs are not stable long-term and must be combined a short time prior to administration.

Reconstitution of a drug or combination of drugs for administration such as reconstitution of a lyophilized drug from its powdered state to a liquid state may require a number of steps such as, for example, mixing the drug with a predetermined amount of a reconstitution liquid (e.g., water) and waiting a minimum time period to allow the reconstitution process to fully complete. It may be beneficial for a device to automate the reconstitution process for ease of use and to reduce the possibility of human error. Furthermore, automated preparation of a drug or drug combination can reduce the risk of exposure to highly toxic or mutagenic substances such as are utilized for chemotherapy. It may also be beneficial for one device to both automatically reconstitute a drug and automatically deliver the reconstituted drug to a patient, thereby further minimizing error and exposure and further increasing ease of use.

### SUMMARY

In one embodiment, a device which automatically reconstitutes and delivers a drug to a user is disclosed. The device comprises a controller; a cartridge; a connector to which a vial removably inserts, wherein inserting the vial into the connector causes the vial to be fluidly coupled to the cartridge through the connector along a first fluid path; a reversible cartridge drive both electrically coupled to the controller such that the controller controls activation of the cartridge drive and mechanically coupled to the cartridge such that activation of the cartridge drive causes fluid to be either transferred into or out of the cartridge; and a second fluid path which is formed following reconstitution to enable a reconstituted drug in the cartridge to be delivered to the user.

In another embodiment, a method for automatically reconstituting a drug is disclosed. The method comprises providing a device which comprises a connector to which a vial removably inserts and also comprises a cartridge, wherein the cartridge can initially be empty, contain a reconstitution liquid or a liquid drug, or contain a solid or semi-solid drug; and inserting the vial into the connector, wherein if the cartridge is initially empty or contains the solid or semi-solid drug the vial contains either the liquid drug or the reconstitution fluid, and wherein if the cartridge initially contains the reconstitution fluid or the liquid drug the vial contains the solid or semi-solid drug, wherein the device automatically prepares a reconstituted drug by transferring the reconstitution liquid or the liquid drug and the solid or semi-solid drug between the vial and the cartridge, and wherein once the reconstitution process is complete the device automatically indicates that the reconstitution process is complete and that the reconstituted drug is contained in the cartridge either ready for delivery or for a new vial to be inserted into the connector.

In still another embodiment, a method for automatically reconstituting a drug is disclosed. The method comprises providing a device for automatically delivering the drug to a user, wherein the device comprises a connector, to which a vial containing the drug removably inserts; placing the device on a substantially flat surface; inserting the vial into the connector, wherein the vial is in a substantially inverted position when inserted; commanding the device to start a reconstitution process to automatically create a reconstituted drug; waiting for the device to indicate that the reconstitution process is complete; removing the vial from the connector; and disposing the device on the user such that the device automatically delivers the reconstituted drug to the user.

In another embodiment, a method for automatically reconstituting and delivering a drug to a user which comprises utilizing the above mentioned device is also disclosed.

In still another embodiment, a method of assembling the above mentioned device is disclosed. The method comprises assembling the cartridge, the connector and the first and second fluid paths as a single module; sterilizing the module; and placing the module into a housing including the controller and cartridge drive to assemble the device.

These and other advantages and novel features of the invention will become apparent from the following detailed description of the invention when considered in conjunction with the drawings.

### BRIEF DESCRIPTION OF THE DRAWINGS

The embodiments set forth in the drawings are illustrative and exemplary in nature and not intended to limit the inventions defined by the claims. The following detailed description of the illustrative embodiments can be understood when read in conjunction with the following drawings, where like structure is indicated with like reference characters and in which:

FIGS. 1A to 1D depict a drug delivery device for a drug such as a lyophilized drug according to one or more embodiments shown and described herein;

FIG. 2 depicts a drug delivery device for a drug such as a lyophilized drug according to one or more embodiments shown and described herein;

FIG. 3 depicts a connector and a vial according to one or more embodiments shown and described herein;

FIG. 4 depicts a vial inserted into a connector according to one or more embodiments shown and described herein;

FIGS. 5A and 5B depict a vial according to one or more embodiments shown and described herein;

FIG. 6 depicts a vial inserted into a connector according to one or more embodiments shown and described herein;

FIG. 7 depicts a drug delivery device for a drug such as a lyophilized drug according to one or more embodiments shown and described herein;

FIG. 8 depicts a connector and a vial according to one or more embodiments shown and described herein;

FIG. 9 depicts a vial inserted into a connector according to one or more embodiments shown and described herein;

FIG. 10 depicts rotation and locking of the vial according to one or more embodiments shown and described herein;

FIGS. 11A to 11F depict rotation of the vial and the cartridge manifold according to one or more embodiments shown and described herein;

FIG. 12 depicts a drug delivery device for a drug such as a lyophilized drug according to one or more embodiments shown and described herein;

FIG. 13 depicts a vial inserted into the drug delivery device according to one or more embodiments shown and described herein;

FIG. 14 depicts a connector and a vial according to one or more embodiments shown and described herein;

FIG. 15 depicts a vial inserted into a connector according to one or more embodiments shown and described herein;

FIG. 16 depicts a vial inserted into a connector according to one or more embodiments shown and described herein;

FIG. 17 depicts movement of the vial according to one or more embodiments shown and described herein;

FIG. 18 depicts the orientation of the injection conduit according to one or more embodiments shown and described herein;

FIGS. 19A to 19D depict a connector and a vial according to one or more embodiments shown and described herein at various stages of operation and interaction therebetween;

FIG. 20 depicts an electrical schematic for a drug delivery device according to one or more embodiments shown and described herein;

FIGS. 21 and 22 each depict a flow diagram of a method for automatically reconstituting a drug, such as for example, a lyophilized drug, according to one or more embodiments shown and described herein;

FIG. 23 depicts a flow diagram of a method for assembling a device which automatically reconstitutes a drug, such as for example, a lyophilized drug, according to one or more embodiments shown and described herein.

### DETAILED DESCRIPTION

For purposes of this disclosure, reconstitution refers to any of and combinations of conversion of a drug in a solid or semi-solid form into a liquid form suitable for administration to an animal by infusion or injection, conversion of a concentrated liquid form of a drug into a diluted liquid form suitable for administration to an animal by infusion or injection, or to preparing a liquid mixture of two or more drugs (each of which can initially be provided in any of a solid, a semi-solid or a liquid form) suitable for administration to an animal by infusion or injection. Thus, a reconstituted drug is any liquid drug formulation formed from any combination of one or more drugs each provided in any of a solid, semi-solid or liquid form and possibly by addition of a reconstitution liquid to dilute or dissolve the drug(s). A reconstitution liquid that is added to one or more drugs to prepare a reconstituted drug can be any suitable liquid pharmaceutical diluent including but not limited to water, buffers, organic solvents such as ethanol and dimethylsulfoxide, and combination thereof. A reconstituted drug can also contain other substances that aid in delivery or increase the stability of the reconstituted drug. For example, a hyaluronidase enzyme that increases the rate of subcutaneous infusion could be part of a reconstituted drug.

For purposes of this disclosure, a lyophilized drug is a drug that has been converted into a powdered form or other suitable form by removing some or all of the moisture contained therein. The drug may be lyophilized by any suitable manner including, but not limited to, a freeze dry process. In order to use the lyophilized drug, it must be reconstituted with a reconstitution liquid such as, for example, water or a buffer. The reconstitution process converts the lyophilized drug into a liquid form so it can be injected or infused into an animal such as a human or a veterinary animal such as a cow, a horse, a sheep, a pig, a dog or a cat.

For purposes of this disclosure, stating that the vial is fluidly coupled to the cartridge means that the content of the vial is fluidly coupled to the content of the cartridge. The content may include liquids, gases, powders, or combinations thereof. For example, the vial may initially contain the lyophilized (i.e., powdered) drug and a gas such as air. As another example, the cartridge may initially contain the reconstitution liquid, which may be water. For purposes of this disclosure, fluid is defined as any material which is capable of flowing such as, for example, air, liquids, powders, and combinations thereof.

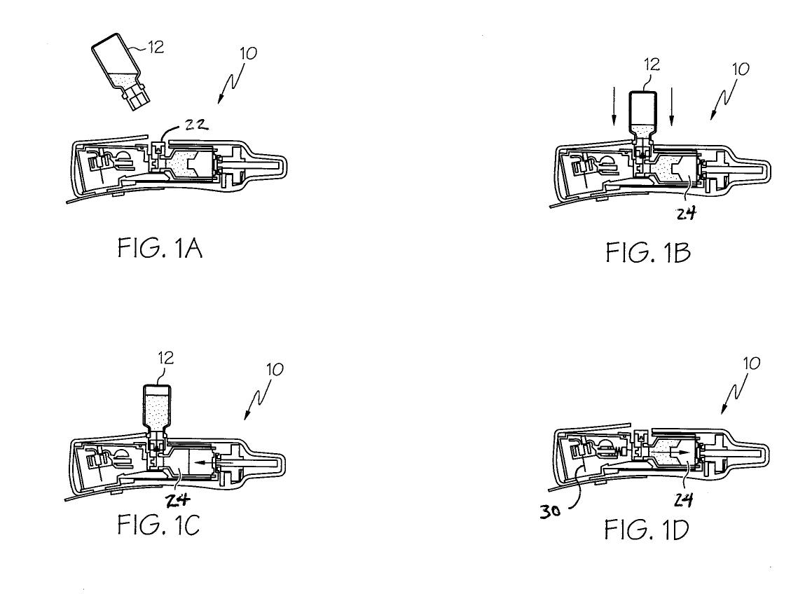

FIGS. 1A to 1D depict one embodiment of a device 10 for automatically reconstituting a drug such as, for example, a lyophilized drug, and automatically delivering the reconstituted drug to a patient. In FIG. 1A, the device 10 is shown as being ready to receive a vial 12 containing the drug. FIG. 1B shows the vial 12 inserted into a connector 22 (FIG. 1A) of the device 10, in which the device 10 includes a cartridge 24 containing the reconstitution liquid. Inserting the vial into the connector 22 of the device 10 causes the vial 12 to be fluidly coupled to the cartridge 24 through the connector 22 along a first fluid path. FIG. 1C shows the reconstitution process, wherein the reconstitution liquid is automatically transferred from the cartridge 24 into the vial 12 along the first fluid path. This causes the drug in the vial 12 to mix with the reconstitution liquid from the cartridge 10c to create the reconstituted drug in the vial 12. The reconstituted drug may be transferred one or more times back and forth between the vial 12 and the cartridge 24 in order to aid in the mixing process. Finally, FIG. 1D shows the reconstituted drug disposed in the cartridge 24 and the vial 12 removed by the user. In addition, the mechanism fluidly couples the reconstituted drug in the cartridge 24 to a second fluid path to enable the reconstituted drug in the cartridge to be delivered to the user. For example, in one embodiment the second fluid path is a delivery path to an injection/infusion needle 30, in which the injection/infusion needle 30 is activated and can then be used to deliver the reconstituted drug to a user. In other embodiments, the second fluid path may be a supply path to a needle-less injector (not shown), which can then be used to deliver the reconstituted drug to the user. After placed on the body of a user, the device 10 may then automatically deliver the reconstituted drug to the user. The construction and operation of the one or more embodiments the device 10 are describe in detail below.

In alternative embodiments, the device is provided with a cartridge that is either empty initially or one that already contains a drug, and in which the drug can be any of a solid drug, semisolid drug or a liquid drug. If the drug in the cartridge is a solid or semi-solid drug, the first vial connected to the device contains either a reconstitution liquid or a liquid drug. Likewise, if the first vial contains a solid or semi-solid drug, the cartridge initially contains a reconstitution liquid or a liquid drug.

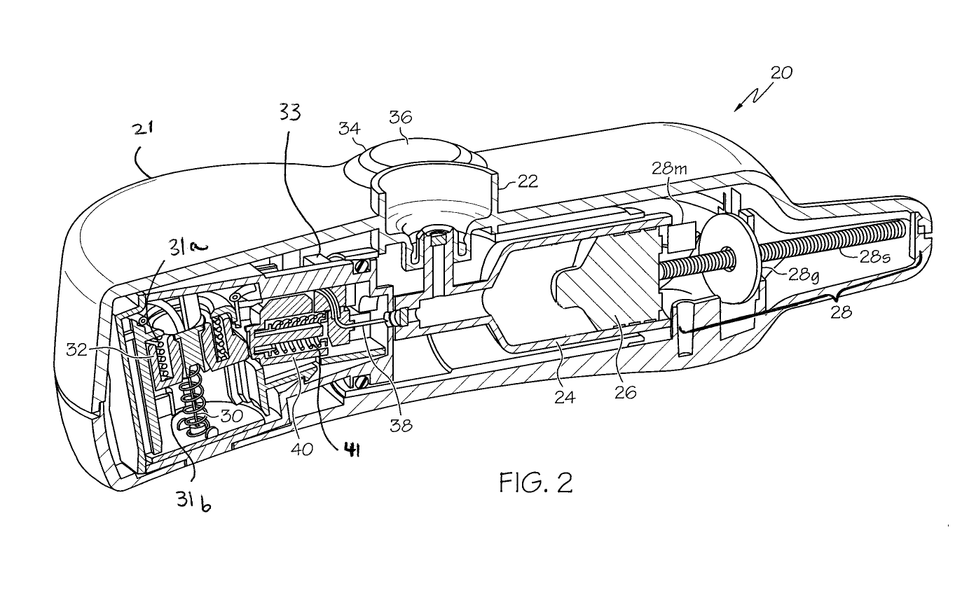

FIG. 2 shows another embodiment of a drug delivery device 20 which is capable of automatically reconstituting a drug such as a lyophilized drug and automatically delivering the reconstituted drug to the user. The device 20 may comprise a connector 22, a cartridge 24 having a plunger 26, a cartridge drive 28, an injection/infusion needle 30, an injection needle drive 32, an annunciator 34, and a user input 36. The connector 22 may be capable of receiving a vial 12 (such as shown by FIGS. 1 and 2) containing the drug. When the vial 12 is inserted into the connector 22, the vial 12 may be fluidly coupled to the cartridge 24. The user may remove the vial 12 from the connector 22 when the reconstitution process has completed.

The cartridge 24 can contain the reconstitution liquid which may be, for example, water. The cartridge 24 may have a plunger 26 disposed therein which is fluidly coupled to the reconstitution liquid. Movement of the plunger 26 changes the pressure of fluid in the cartridge 24 to cause fluid to be transferred into or out of the cartridge 24. For example, when the vial is inserted into the connector 22, the vial may be fluidly coupled to the fluid-containing cartridge 24. In this example, applying pressure to the fluid (e.g., the reconstitution liquid) in the cartridge 24 (by the plunger 26) causes fluid in the cartridge 24 to be transferred to the vial. Likewise, removing pressure from the fluid in the cartridge 24 causes the fluid in the vial to be transferred to the cartridge 24. In this manner, movement of the plunger 26 causes fluid to be transferred into or out of the cartridge 24.

The cartridge 24 may be cylindrical or have any other suitable geometric shape. In the embodiment shown in FIG. 2, the cartridge 24 is cylindrical and has two portions with different diameters. The portion in which the plunger is disposed has a relatively large diameter, and the portion proximate to the connector 22 has a relatively small diameter. It is contemplated that other suitable shapes and sizes of the cartridge 24 may be used as well, for example, cartridges with oval or substantially rectangular cross-sections. The plunger 26 is disposed in the cartridge 24 and is capable of moving along a longitudinal axis of the cartridge 24. Movement of the plunger 26 causes pressure to change on the fluid in the cartridge 24 and causes fluid to be transferred into or out of the cartridge 24. The plunger 26 may be substantially cylindrical, as shown in FIG. 2, and may also conform to the shape of the cartridge 24 so that, when the plunger 26 is fully extended into the cartridge 24, most or all of the fluid in the cartridge 24 is transferred out of the cartridge 24 (e.g., transferred into the vial or delivered to the user). The plunger 26 may include o-rings which facilitate the sealing of the plunger 26 against the wall of the cartridge 24.

The cartridge drive 28 may comprise a motor 28m, a gear 28g, and a lead screw 28s. The cartridge drive 28 may be mechanically coupled to the plunger 26 such that cartridge drive 28 is capable of moving the plunger 26 in order to adjust the pressure of fluid in the cartridge 24. Therefore, activation of the cartridge drive 28 causes fluid to be transferred into or out of the cartridge 24. The motor 28m may be electrically coupled to a controller (not shown) which controls the activation of the motor 28m. The motor 28m may be mechanically coupled to the gear 28g, and the gear may be mechanically coupled to the lead screw 28s. Rotation of the motor 28m causes the lead screw 28s to move in a linear motion. The lead screw 28s may be mechanically coupled to the plunger 26. Thus, activation of the motor 28m causes movement of the plunger 26, which causes fluid to the transferred into or out of the cartridge 24.

The injection/infusion needle 30 is capable of being fluidly coupled to the user. In FIG. 2, the injection/infusion needle 30 is shown in its deactivated state in which the injection/infusion needle 30 is fully contained within the housing 21 of the device 20. When activated, the injection/infusion needle 30 automatically protrudes from the device 20 such that it is capable of entering the body of the user. When this occurs, the reconstituted drug in the cartridge 24 may be fluidly coupled to the user such that the activation of the cartridge drive 28 causes the reconstituted drug to be delivered to the user. The injection/infusion needle 30 may comprise stainless steel or other suitable material. The injection/infusion needle 30 may be fluidly coupled to the cartridge 24 via a cartridge needle 38. In some embodiments, the cartridge 24 can be moved in a first direction by the cartridge drive 28 (via movement of plunger 26) to insert the cartridge needle 38 into the cartridge 24.

The injection needle drive 32 may be mechanically coupled to the injection/infusion needle 30 such that the injection needle drive 32 is capable of inserting the injection/infusion needle 30 into a patient, which patient can be the user. In one embodiment, the injection needle drive 32 may comprise a pair of springs, in which a first spring 31a is releasably provided by a catch (not shown) under compression and a second spring 31b is provided not under compression. In such an embodiment, when the first spring 31a is released via movement of the catch, e.g., automatically by operation of a controller 33 of the device or manually, the injection/infusion needle 30 is extended outwardly from the housing 21 of the device 20 by the expansion of the first spring 31a for the purpose of being driven into the user's body, and also compresses the second spring 31b. After expansion of the first spring 31a, the same or another catch (not shown) holds releasably the second spring 31b under compression. Upon release of the catch holding the second spring 31b, e.g., automatically by operation of the controller 33 of the device or manually, such as after completion of infusing the drug into the user, the injection/infusion needle 30 is retracted back into the housing 21 of the device 20 by the expansion of the second spring 31b. In another embodiment, the injection needle drive 32 may comprise an electrically operated solenoid as a replacement to the first spring 31a. The second spring 31b in this embodiment is operatively coupled to the injection/infusion needle 30 such that the force of the spring keeps the injection/infusion needle 30 disposed inside the housing 21 of the device 20 when the solenoid is not activated. The solenoid may be mechanically coupled to the injection/infusion needle 30 such that, when activated, it opposes the force of the second spring 31b and causes the injection/infusion needle 30 to extend from the housing 21 and be inserted into the body of the user. The solenoid may be activated by passing a current through the solenoid. For example, the controller 33 may be electrically coupled to the solenoid and control whether the solenoid is activated or deactivated. The injection needle drive 32 may comprise other types of actuators, as is known in the art.

The annunciator 34 may comprise a light, a light emitting diode (LED), or other suitable device. The annunciator 34 may be electrically coupled to the controller 33 such that the controller controls the activation of the annunciator 34. For example, if the annunciator 34 is a light, the controller 33 controls whether the annunciator 34 is activated (e.g., illuminated) or deactivated (e.g., extinguished). The annunciator 34 may comprise other types of devices such as, for example, acoustic devices, vibratory devices, or combinations thereof. In the embodiment shown in FIG. 2, the user input 36 is a pushbutton, and the annunciator 34 is an LED which surrounds the pushbutton, such as for example, as an annular ring. The annunciator 34 may indicate to the user the status of the drug delivery device 20. For example, the annunciator 34 may flash when the drug has been reconstituted and the vial is ready to be removed from the device. The annunciator 34 may also indicate other status information such as, for example, whether an error occurred during the reconstitution process, whether the battery is low, etc.

The user input 36 may comprise a pushbutton, a switch, or other suitable device. The user input 36 may be electrically coupled to the controller 33 such that the controller is able to determine whether the user is activating the user input 36. In one embodiment, the user may insert the vial into the connector 22 and activate (e.g., press) the user input 36 to inform the controller that the reconstitution process is ready to begin. The user input 36 may comprise other types of devices as well. In one embodiment (not shown), the device further comprises a sensor (mechanical, electromechanical, optical etc.) that detects the proximity of the device to the body of a patient and only when and during the time the device is held near to the patient (such as held on by an adhesive layer on the bottom portion of the device) is the device able to be activated and/or continue to deliver a drug to the patient. Furthermore, when the proximity sensor detects loss of contact with the body of a patient during drug delivery it is also possible for the device to retract (immediately or with a time delay) a delivery device (for example, an infusion or injection needle) such that danger of contact with a sharp and possibly contaminated needle is reduced.

The cartridge needle 38 may be fluidly coupled to the injection/infusion needle 30. The cartridge needle 38 may also be mechanically coupled to the cartridge needle drive 40 such that the cartridge needle drive 40 is capable of inserting the cartridge needle 38 into the cartridge 24. In one embodiment, the cartridge needle drive 40 comprises a spring 41 that is releasably provided by a catch (not shown) under compression. In such an embodiment, when the spring 41 is released via movement of the catch, e.g., automatically by operation of the controller 33 of the device or manually, the cartridge needle 38 is inserted into and fluidly coupled to the cartridge 24. In another embodiment, the cartridge needle drive 40 may comprise a solenoid which replaces the spring 41. In such an embodiment, the solenoid may be mechanically coupled to the cartridge needle 38 such that, when activated, it inserts into and fluidly couples the cartridge needle 38 to the cartridge 24 and remain fluidly coupled when deactivated in one embodiment, or decoupled therefrom when deactivated in another embodiment via a biasing force from a return spring (not shown). The solenoid may be activated by passing a current through the solevoid. For example, controller 33 may be electrically coupled to the solenoid and control when to activate and deactivate the solenoid. The cartridge needle drive 40 may comprise other types of actuators, as is known in the art. Alternatively, or in combination with a needle drive, a cartridge drive that moves the cartridge to connect the cartridge with the cartridge needle can be employed.

Referring still to FIG. 2, the basic operation of the device 20 is now described. The device 20 may operate such that the user initially places the device 20 on a relatively flat surface. The controller 33 of the device 20 waits for the user to indicate via the user input 36 that the user has inserted the vial into the connector 22 (e.g., as shown by FIG. 1B). Alternatively, the device can automatically detect the vial such as by a contact, optical or Hall Effect sensor. Furthermore, the time and sequence of reconstitution steps can either be set in the device by default or can be set prior to use by virtue of another information source such as an identification label on a vial containing a drug or a vial containing a reconstitution liquid (for example, a bar-code, an OCR code, an RFID-Tag, a mechanical code or a contact code). In addition, the device can, based on the information contained in such codes or user input, guide a user through a complicated reconstitution procedure (such as where multiple drug/reconstitution liquid vials are loaded into the device in a particular order). To further ensure that a complicated series of vial connections and reconstitution steps is carried out properly, the device can further check that the correct vial is in place at a given point in a reconstitution procedure (such as by reading a barcode or any other information source/identifier on the vial) and time periods for reconstitution steps are followed. An alarm or alarms (audible, tactile and/or visual) can be employed to alert a user to change vials at the correct time, and/or to warn the user of incorrect vial insertions and/or attempts by a user to remove a vial prior to a reconstitution step being completed.

After receiving indication via the user input 36 that the vial is inserted into the connector 22, the controller 33 automatically transfers, by activating the cartridge drive 28 in a first manner, the reconstitution liquid out of the cartridge 24, through the connector 22, and into the vial containing the drug to create a reconstituted drug (e.g., as shown by FIG. 1C). Activating the cartridge drive 28 in the first manner may comprise activating the motor 28m to cause the plunger 26 to move in a direction to increase the pressure on the reconstituted liquid in the cartridge 24. The controller 33 automatically transfers the reconstituted drug out of the vial, through the connector 22, and into the cartridge 24, by activating the cartridge drive 28 in a second manner. Activating the cartridge drive 28 in the second manner may comprise activating the motor 28m to cause the plunger 26 to move in a direction to decrease the pressure on fluid in the cartridge 24 (e.g., opposite to the first manner as shown by FIG. 1D), thus causing fluid to be transferred from the vial to the cartridge 24.

The controller 33 may then automatically activate the annunciator 34 to indicate to the user that the reconstituted drug is disposed in the cartridge 24 (e.g., as also shown by FIG. 1D) and the vial is ready to be removed from the connector 22. The controller 33 waits for the user to indicate via the user input 36 that the vial is removed from the connector 22 and that the device 20 is physically coupled (removably) to the user. Once the device 20 is coupled to the user (e.g., via an adhesive or plaster), the user operates the user input 36 to provide such indication, wherein the controller 33 then fluidly couples automatically the injection/infusion needle 30 to the cartridge 24. This may be accomplished by the controller 33 activating the cartridge needle drive 40 which causes the cartridge needle 38 to be inserted into and fluidly coupled to the cartridge 24 (and/or a cartridge driver moves the cartridge to couple it with the cartridge needle). The controller 33 also fluidly couples automatically the injection/infusion needle 30 to the user by activating the injection needle drive 32. Finally, the controller 33 delivers automatically the reconstituted drug from the cartridge 24, through the injection/infusion needle 30, and to the user, by activating the cartridge drive 28 in the first manner. As discussed above, activating the cartridge drive 28 in the first manner may comprise activating the motor 28m to cause the plunger 26 to move in a direction to increase the pressure on the reconstituted drug in the cartridge 24. These steps may be performed in any suitable order.

The controller 33 may perform additional steps which facilitates the reconstitution of the drug. For example, the controller 33 may agitate the reconstituted drug to speed up the mixing of its constituents (e.g., the lyophilized drug and the reconstitution liquid). This may be accomplished by the controller 33 automatically transferring the reconstituted drug out of the cartridge 24, through the connector 22, and into the vial by activating the cartridge drive 28 in the first manner. The controller 33 may then automatically transfer the reconstituted drug out of the vial, through the connector 22, and into the cartridge 24 by activating the cartridge drive 28 in the second manner. This transferring of the reconstituted back and forth between the cartridge 24 and the vial may be performed one or more times and may cause the reconstituted drug to be mixed more thoroughly.

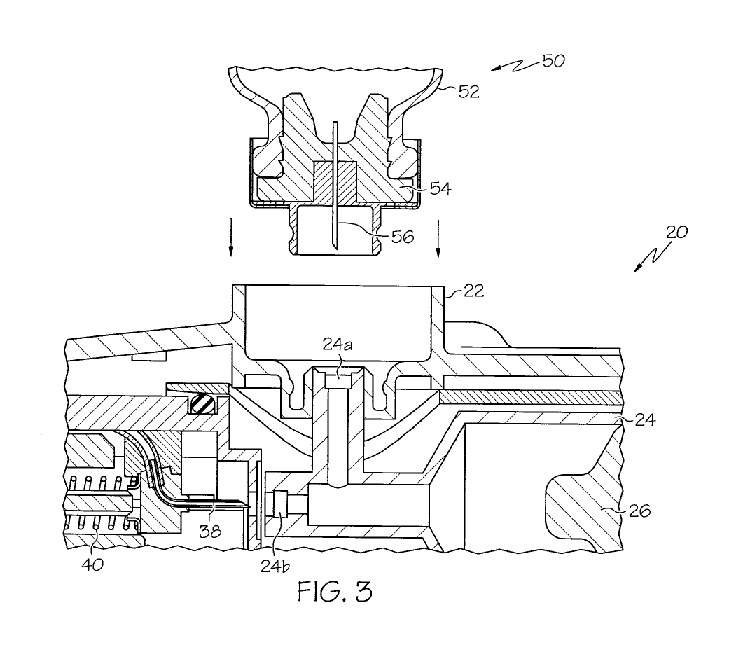

The device 20 may further comprise a sensor (not shown) which is electrically coupled to the controller 33 and mechanically coupled to the device 20. The sensor may sense whether the device 20 is disposed on a surface and may transmit an electrical signal to the controller 33 indicating whether the device 20 is disposed on the surface. The surface sensor could be the same or different from the body proximity sensor discussed above. If the device 20 is not disposed on a surface, the controller 33 may automatically inhibit transferring the reconstitution liquid out of the cartridge 24 and into the vial containing the drug. This may require the user to place the device 20 on a relatively flat and level surface before beginning the reconstitution process. Similarly, when the device 20 is disposed on a surface, the device 20 may be configured such that, when the vial is inserted into the connector 22, the vial must be substantially inverted. For purposes of this disclosure, “substantially inverted” means that the vial is oriented such that an opening of the vial is gravitationally lower than the body of the vial. Alternatively, an orientation sensor could ensure that the vial is substantially inverted without also checking whether it is on a surface. FIG. 3 depicts a vial 50 being inserted into the connector 22 of the device 20 from FIG. 2. The vial 50 may comprise a body 52, a stopper 54, and a vial conduit 56. The stopper 54 may be inserted into a neck of the vial 50 and may seal the drug (disposed in the body 52) in the vial 50. The vial conduit 56 may be disposed in and mechanically coupled to the stopper 54 such that the vial conduit 56 and the stopper 54 move in unison. As shown in the embodiment in FIG. 3, the vial conduit 56 may pass through the stopper 54 so that it is fluidly coupled to the drug contained within the vial 50. The vial 50 may have a cover (not shown) which is disposed over the vial conduit 56 to protect the vial conduit 56 and keep the vial conduit 56 (and the drug) aseptic.

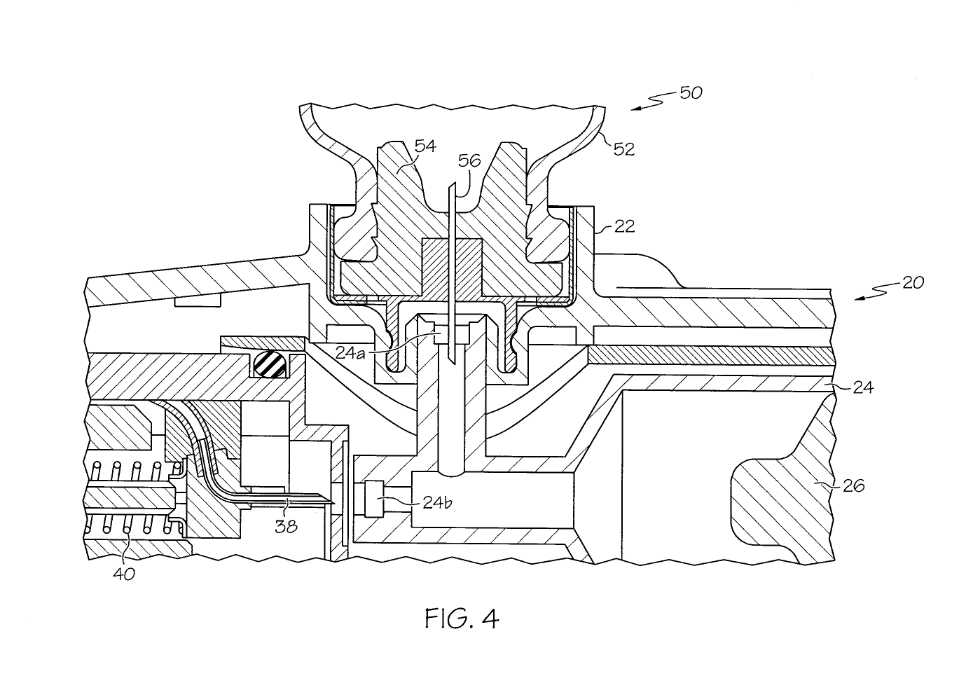

Referring to FIGS. 3 and 4, the vial conduit 56 may comprise a needle of a suitable material such as, for example, stainless steel. The needle may be hollow to provide a fluid conduit between the vial 50 and the cartridge 24 when the vial is inserted into the connector 22. The cartridge 24 may include a septum 24a which is capable of being pierced by the vial conduit 56 when the vial 50 is inserted in the connector 22. The septum 24a may comprise a rubber, plastic, or other suitable material. The septum 24a may be capable of re-sealing itself when the vial conduit 56 is removed from the septum 24a (e.g., when the vial 50 is removed from the connector 22). In this embodiment, when the user inserts the vial 50 in the connector 22, the vial conduit 56 pierces the septum 24a and fluidly couples the vial 50 to the cartridge 24. In addition, when the user removes the vial 50 from the connector 22, the vial conduit 56 is removed from the septum 24a which may re-seal itself so that the cartridge 24 is fluidly uncoupled (at least through the septum 24a). It is contemplated that other techniques may be used to couple fluidly the vial 50 to the cartridge 24.

The cartridge 24 may include an additional septum 24b which permits the cartridge needle 38 to be fluidly coupled to the cartridge 24 when the cartridge needle drive 40 is activated. The additional septum 24b may operate similarly to the septum 24a, described above. That is, the additional septum 24b may be capable of being pierced by the cartridge needle 38 when the cartridge needle drive 40 is activated. The additional septum 24b may comprise a rubber, plastic, or other suitable material. The additional septum 24b may also be capable of re-sealing itself, which is useful in the embodiment of the device 20 in which the cartridge needle drive 40 comprises a solenoid such that when the cartridge needle drive 40 (i.e., solenoid) is deactivated, the cartridge needle 38 retracts as previously described above. In this embodiment, the controller 33 automatically controls whether the cartridge needle drive 40 is activated or deactivated. It is contemplated that other techniques may be used to couple fluidly the cartridge needle 38 to the cartridge 24.

Generally, either the vial 50 or the cartridge needle 38 is fluidly coupled to the cartridge 24 at a particular instant in time. For example, when the device 20 is reconstituting the drug, the vial 50 is fluidly coupled to the cartridge 24 (via septum 24a) and the cartridge needle 38 is not fluidly coupled to the cartridge 24. In addition, when the device is delivering the reconstituted drug to the user, the cartridge needle 38 (and, hence, the injection needle) are fluidly coupled to the cartridge 24 (via additional septum 24b) and the vial 50 is fluidly uncoupled from the cartridge 24 (e.g., by removal of the vial 50 from the connector 22). Through the manual actions of the user and the automatic actions of the controller 33, the device 20 controls the automatic reconstitution of the drug and the automatic delivery of the reconstituted drug to the user.

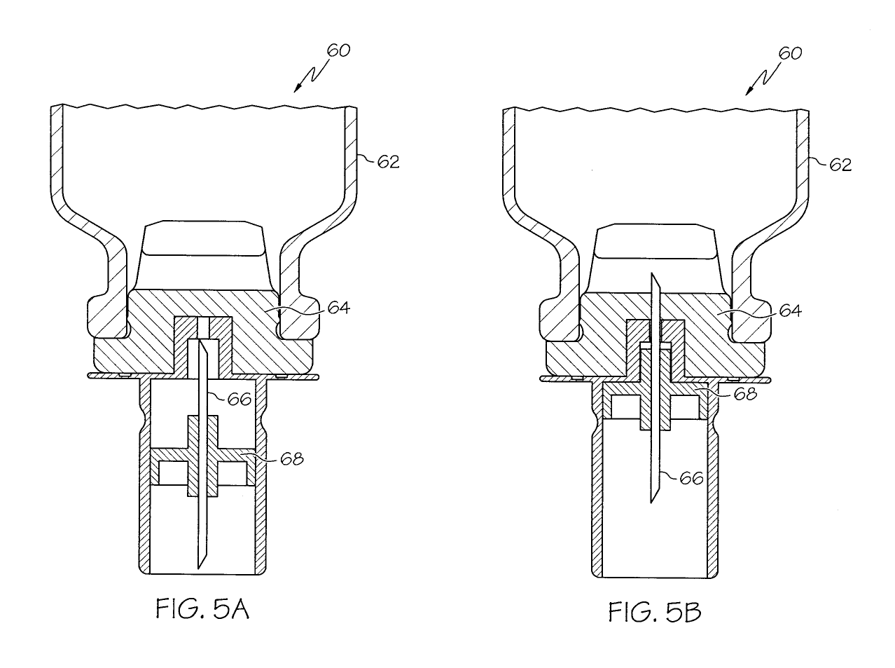

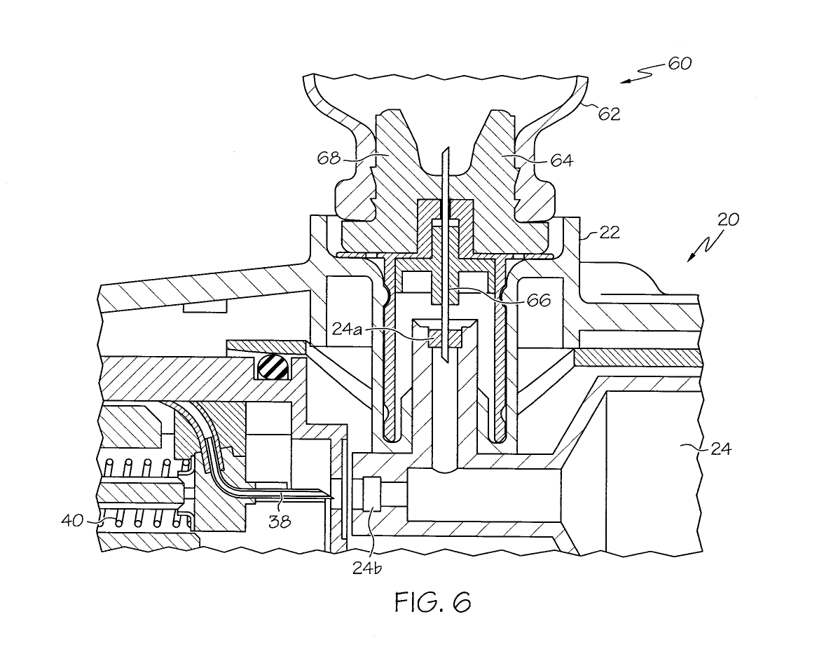

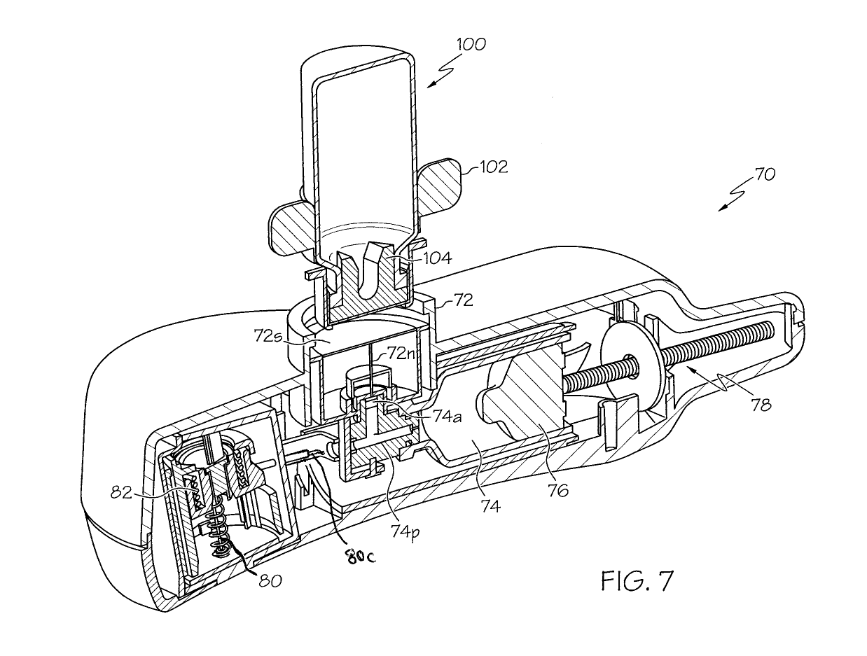

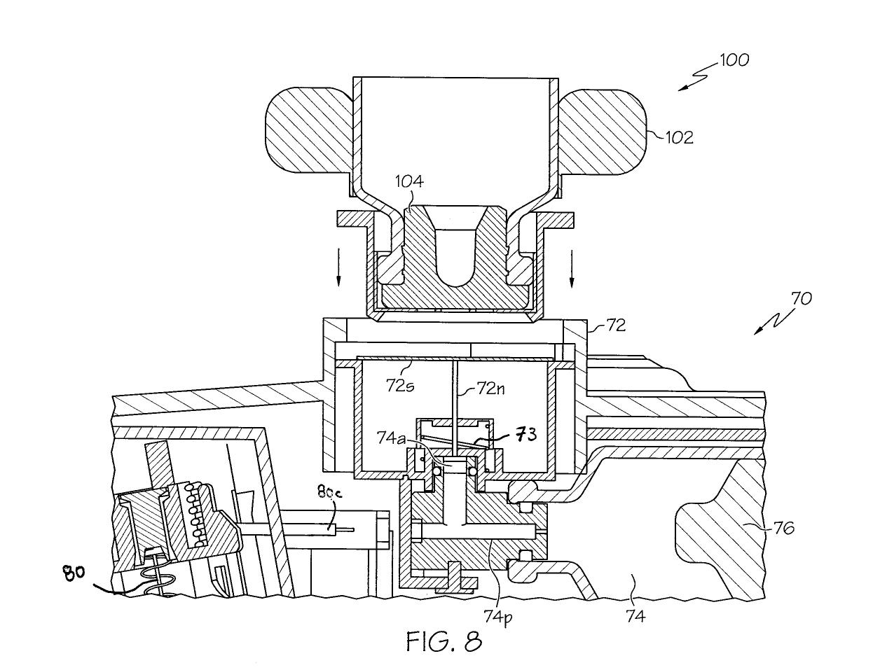

FIGS. 5A, 5B, and 6 show another embodiment of a vial 60 which may be inserted into the connector 22 of the drug delivery device 20. In this embodiment, the vial 60 includes a body 62, a vial septum 64, a shuttle needle 66, and a guide 68. The shuttle needle 66 may initially not be fluidly coupled to the drug (as in the embodiment shown in FIGS. 3 and 4). The shuttle needle 66 may be mechanically coupled to the guide 68 such that they move in unison in the vial cap in a direction substantially parallel to the longitudinal axis of the shuttle needle 66. As shown in FIG. 5A, before the vial 60 is inserted into the connector 22, the shuttle needle 66 has not pierced the vial septum 64. FIG. 5B shows the disposition of the shuttle needle 66 after the vial 60 is inserted in connector 22. The action of inserting the vial 60 into the connector causes the guide 68 to be moved in a direction which causes the shuttle needle 66 to pierce the vial septum 64 and fluidly coupled the shuttle needle 66 to the drug. FIG. 6 depicts the vial 60 fully inserted in the connector 22 in which the shuttle needle 66 has also pierced the cartridge septum 24a of the cartridge 24. In this embodiment, the connector 22 may have a suitable structure such as, for example, a collet which presses against the guide 68 when the vial 60 is inserted in the connector 22, which causes the shuttle needle 66 to pierce both the vial septum 64 and the septum 24a. The shuttle needle 66 may comprise, for example, a hollow stainless steel needle or other suitable device. Another embodiment of a drug delivery device is discussed hereafter with reference made to FIGS. 7-11.

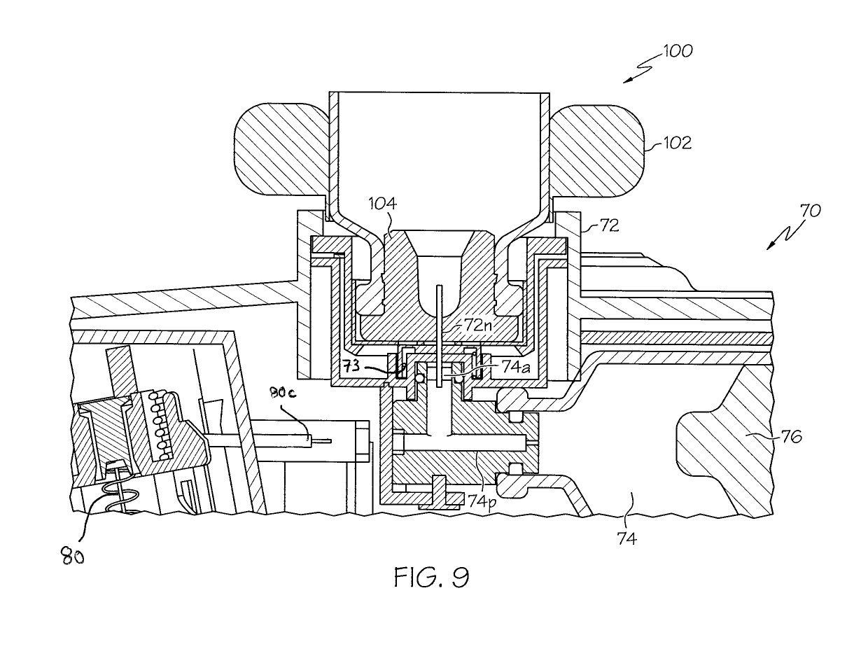

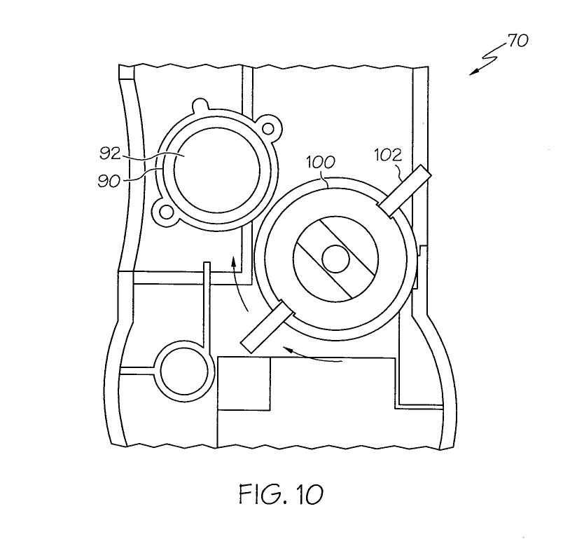

FIG. 7 depicts another embodiment of a drug delivery device 70 which comprises a connector 72, a cartridge 74 having a plenum or cartridge conduit 74p and a plunger 76, a cartridge drive 78, an injection needle 80, and an injection needle drive 82. The cartridge conduit 74p is provided to fluidly connect and distribute the content of the vial 100 to the cartridge and/or vice versa, as well as the contents of the cartridge 74 to the injection needle 80 via an injection conduit 80c such that the content of the cartridge 74 can be delivered to a patient via the injection needle 80. FIG. 10 also depicts the device 70 as having an annunciator 90 and a user input 92. The cartridge 74, plunger 76, cartridge drive 78, injection needle 80, injection needle drive 82, annunciator 90, and user input 92 may operate as previously described with respect to the embodiment of FIGS. 1 to 6. In the embodiment of FIG. 7, the connector 72 may be rotatable about a longitudinal axis of the connector 72 and may be configured to receive the vial 100 when the vial 100 is inserted into the connector 72. The vial 100 may include ears 102 and a stopper 104. When the vial 100 is inserted into the connector 72, the vial is mechanically coupled to the connector 72 such that rotation of the vial 100 (e.g., by the user rotating the vial 100 via the ears 102) causes the connector 72 to rotate in unison. Greater details regarding this rotation in which to cause a fluid connection between the content(s) of the cartridge 74 and the vial 100 as well as the content of the cartridge 74 and the injection needle 80 are provided hereafter in later sections with reference made to FIGS. 9, 10 and 11A-F.

FIG. 8 depicts the vial 100 being inserted into the connector 72 of the device 70 from FIG. 7. The connector 72 of the drug delivery device 70 may have a cover 72s and a vial conduit 72n. The cover 72s may comprise paper, plastic, or other suitable material and may seal the connector 72 to keep unwanted dirt or germs out of the connector 72 before the device 70 is ready to be used, and maintain the sterility of the connector and cartridge. When the vial 100 is inserted in the connector 72 by the user, the cover 72s may simply tear or give way under the force of the vial 100. The cover 72s may also conceal and protect the vial conduit 72n which is disposed in the connector 72 and is capable of piercing the stopper 104. The stopper 104 may be inserted into a neck of the vial 100 to seal the drug (disposed in the body) in the vial 100. The stopper 104 may comprise rubber, plastic, or other suitable material and may be configured to be pierced by the vial conduit 72n.

Referring to FIGS. 8 and 9, when the vial 100 is inserted into the connector 72, the vial conduit 72n pierces the stopper 104 of the vial 100 and causes the vial conduit 72n to be fluidly coupled to the vial 100 (and the drug contained therein). The vial conduit 72n may be mechanically coupled to a spring 73 such that the vial conduit 72n is disposed proximate to a septum 74a of the cartridge 74. When the vial 100 is fully inserted into the connector 72, the spring 73 may be compressed such that the vial conduit 72n pierces the septum 74a, thereby fluidly coupling the vial 100 to the cartridge 74. When the vial 100 is removed from the connector 72, the user pulls the vial 100 from the connector 72 which pulls the vial conduit 72n from the vial 100 and also causes the spring 73 to force the vial conduit 72n out of the septum 74a, thereby fluidly uncoupling the vial conduit 72n from the cartridge 74.

Referring to FIGS. 9 and 10, when the vial 100 is inserted into the connector 72, the vial 100 may be mechanically coupled to the connector 72 such that they rotate in unison about a longitudinal axis of the connector 72 as mentioned above previously. When the vial 100 is inserted into the connector 72, rotation of the vial 100 in a first rotational direction by a locked angle amount causes the vial 100 to be locked in the connector 72. This may prevent the vial 100 from being removed during the reconstitution process and hold the vial 100 in position against the force of the spring 73. The locked angle amount may be about 45°, as shown in FIG. 10. After the drug has been reconstituted, the vial 100 may be rotated in the first rotational direction or in a second rotational direction by the locked angle amount in order to be removed from the connector 72.

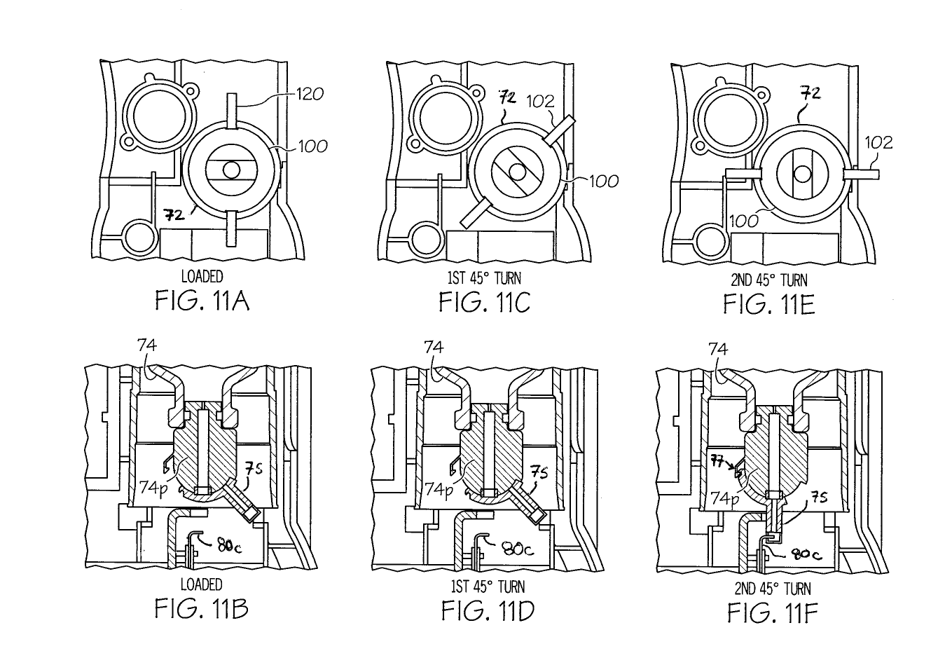

Referring to FIGS. 9 and 11A to 11F, the connector 72 may be mechanically coupled to the cartridge conduit 74p which is capable of fluidly coupling the cartridge 74 to the injection needle 80 as discussed hereafter. FIG. 11A shows the vial 100 after it is initially inserted (i.e., loaded) into the connector 72. In the position of FIG. 11A, the cartridge conduit 74p is disposed away from the injection conduit 80c so that the injection needle 80 (FIG. 9) is not fluidly coupled to the cartridge 74 as depicted by FIG. 11B. FIG. 11C shows the vial 100 after it is rotated by the user by the lock angle amount (e.g., about 45° in this figure). This rotation locks the vial 100 in place and prevents it from being removed from the connector 72. The cartridge conduit 74p is still disposed away from the injection conduit 80c as depicted by FIG. 11D. FIG. 11E shows the vial 100 after it is rotated again by the user by the lock angle amount. This rotation causes a rotatable portion 75 of the cartridge conduit 74p to rotate and be disposed in a position to couple fluidly the cartridge 74 to the injection conduit 80c as depicted by FIG. 11F. A locking arrangement 77 may be provided between the rotatable portion 75 and the cartridge conduit 74p to ensure that the rotatable portion 75 does not rotate when the cartridge 74 is fluidly coupled to the injection conduit 80c. In this locked position the injection needle 80 is then fluidly coupled to the cartridge 74, such that the device 70 may deliver the reconstituted drug to a patient.

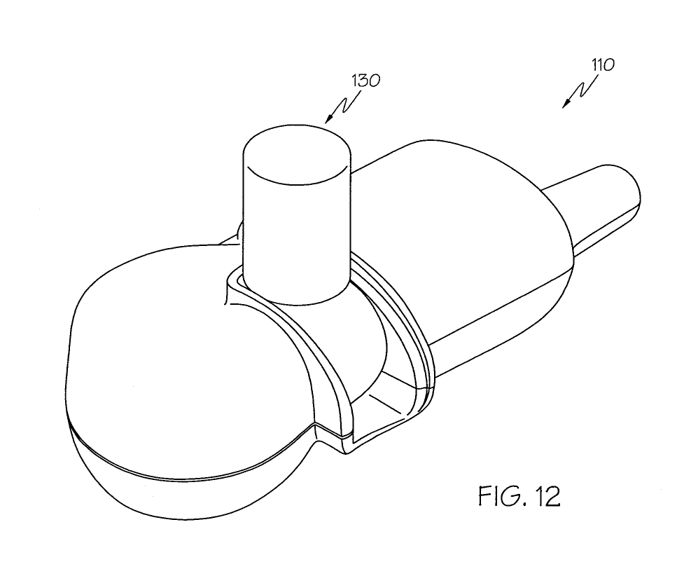

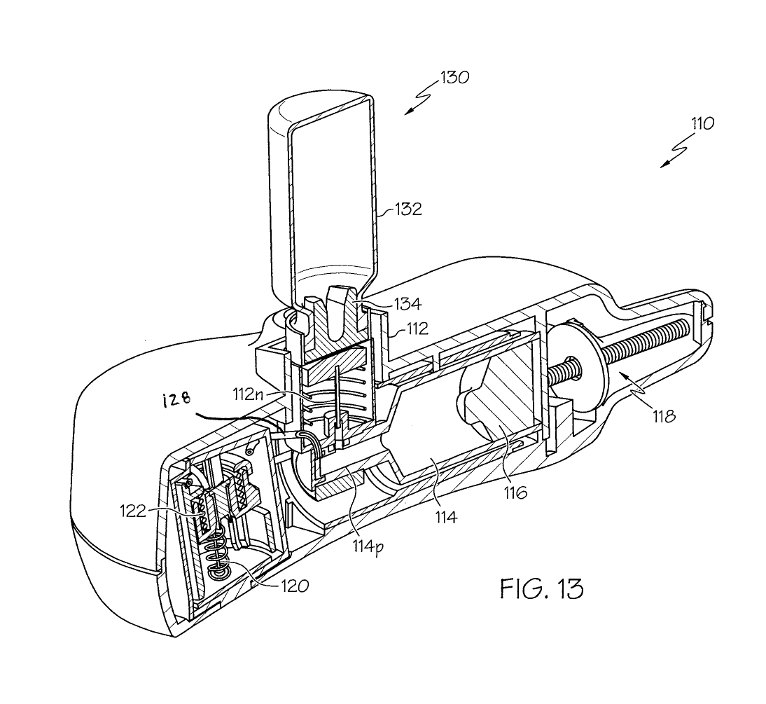

FIG. 12 depicts yet another embodiment of a drug delivery device 110 in which a vial 130 containing the drug may be inserted. The device 110 may automatically reconstitute the drug and automatically deliver the drug to the user. Referring to FIG. 13, this embodiment of the device comprises a connector 112 having a vial conduit 112n, a cartridge 114 having a plenum 114p and a plunger 116, a cartridge drive 118, an injection needle 120, an injection needle drive 122, an injection needle conduit 128, an annunciator (not shown), and a user input (not shown). The cartridge 114, plunger 116, cartridge drive 118, injection needle 120, injection needle drive 122, annunciator, and user input may operate as previously described herein.

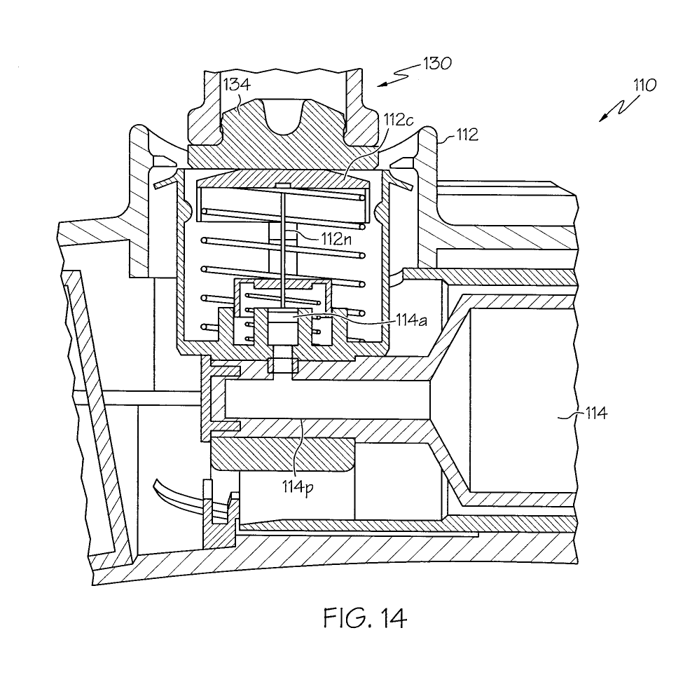

Referring to FIG. 14, the vial 130 is shown being inserted into the connector 112. The vial 130 may comprise a stopper 134 which may comprise rubber, plastic, or other suitable material.

The stopper 134 may be capable of being pierced by the vial conduit 112n which is disposed in the connector 112. The cartridge 114 may comprise a septum 114a which is also capable of being pierced by the vial conduit 112n. The vial conduit 112n may be disposed in the connector 112 and may be mechanically coupled to a spring. The spring may force the vial conduit 112n away from the septum 114a when the vial 130 is not inserted in the connector 112. The connector 112 may also comprise a cover 112c which may be mechanically coupled to an additional spring which forces the cover 112c to seal the opening of the connector 112 when the vial 130 is not inserted in the connector 112. The cover 112c may comprise rubber, plastic, or other suitable material, and may be capable of being pierced by the vial conduit 112n when the vial 130 is inserted in the connector 112.

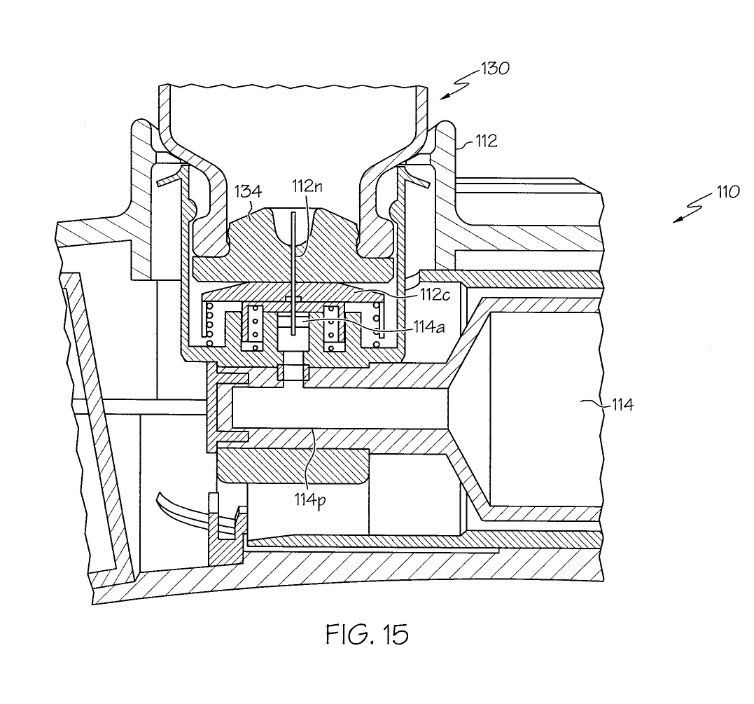

FIG. 15 shows the vial 130 after it is inserted into the connector 112. When the vial 130 is inserted into the connector 112 by the user, the force of the insertion causes the vial conduit 112n to pierce the cover 112c and the stopper 134, thereby fluidly coupling the vial conduit 112n to the vial 130 (and the drug contained therein). The insertion of the vial 130 causes the spring mechanically coupled to the vial conduit 112n and the additional spring mechanically coupled to the cover 112c to compress, thus allowing the vial conduit 112n to pierce the cover 112c and the stopper 134. At the same time, the insertion force of the vial 130 causes the vial conduit 112n to pierce the septum 114a, thus causing the vial conduit 112n to be fluidly coupled to the cartridge 114. In this manner, inserting the vial 130 into the connector 112 causes the vial 130 to be fluidly coupled to the cartridge 114. Once the vial 130 is inserted into the connector 112, the user may activate the user input (not shown) to start the automatic reconstitution process. During the reconstitution process, the controller 33 (FIG. 2) may cause the cartridge drive (e.g. see the cartridge drive 118 of FIG. 13) to transfer the reconstitution liquid out of the cartridge 114 and into the vial 130 through the connector 112 (and through the vial conduit 112n) to create a reconstituted drug through the techniques shown and described herein. The controller 33 may then cause the cartridge drive to transfer the reconstituted drug out of the vial 130 and into the cartridge 114 through the vial conduit 112n. The controller 33 may then inform the user that the reconstitution process has completed by activating the annunciator.

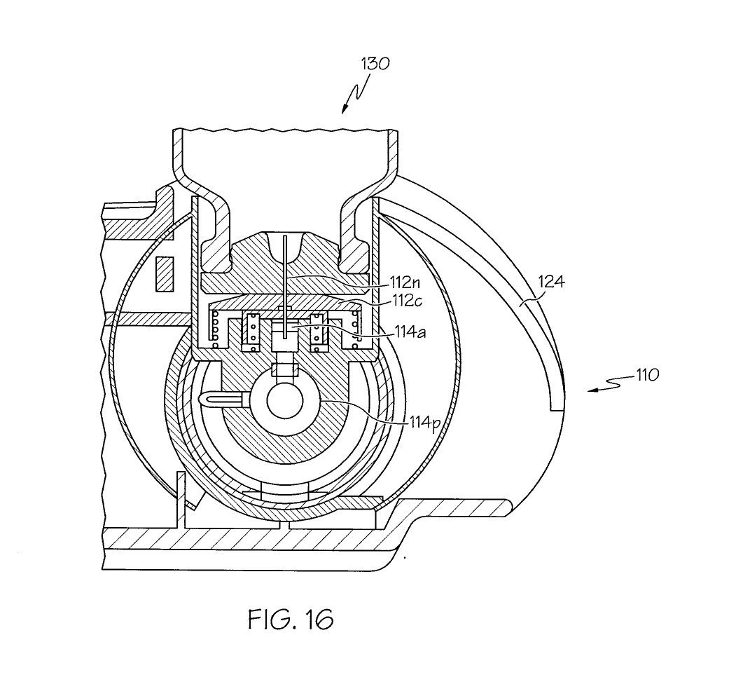

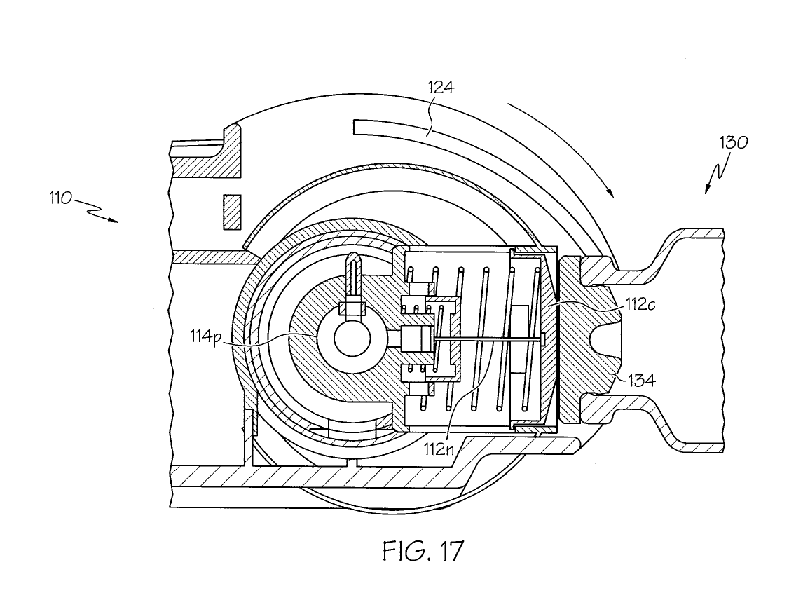

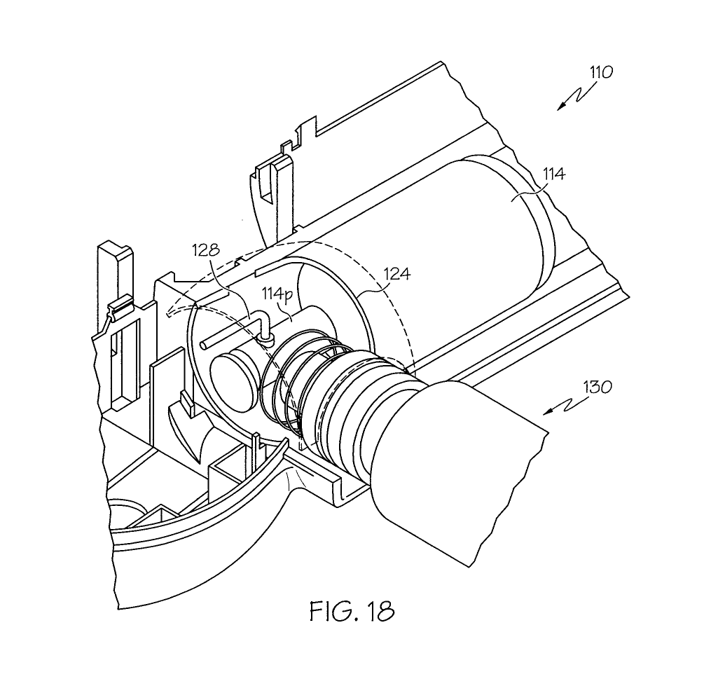

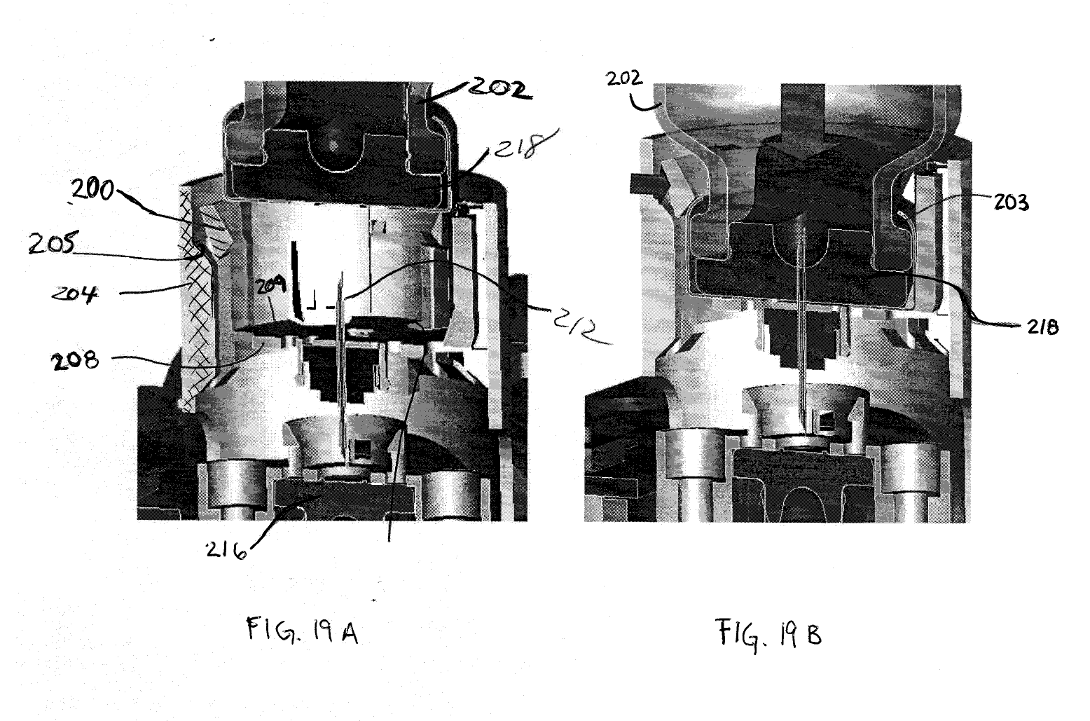

FIGS. 16 to 18 depict the removal of the vial 130 from the connector 112 by the user. The connector 112 may be capable of rotating about a longitudinal axis of the cartridge 114. The housing of the device 110 may include an arcuate rail 124 which is mechanically coupled to the vial such that, when the vial 130 rotated from a first orientation (e.g., the orientation shown in FIG. 16) to a second orientation (e.g., the orientation shown in FIG. 17), the arcuate rail 124 engages the vial 130 and forces the vial 130 out of the connector 112. When the user is ready to remove the vial 130 from the connector 112, the user rotates the vial 130 about the longitudinal axis of the cartridge 114, which causes the vial 130 to be fluidly decoupled from the connector 112 by the action of the arcuate rail 124. When the user has rotated the vial 130 into a substantially horizontal position, as shown in FIG. 17, the vial 130 has been forced away from the connector 112, and the vial conduit 112n has been correspondingly fluidly decoupled from both the vial 130 and the cartridge 114. In addition, when the vial 130 has been rotated into the horizontal position by the user, the injection needle 120 (FIG. 13) is fluidly coupled to the cartridge 114 via the injection needle conduit 128. As shown in FIG. 18, the injection needle conduit 128 is aligned with the plenum 114p such that the injection needle conduit 128 is fluidly coupled to the plenum 114p and, consequently, to the cartridge 114. After rotating the vial 130 into the substantially horizontal position (as shown in FIG. 18), the user may remove the vial 130 from the device 110.

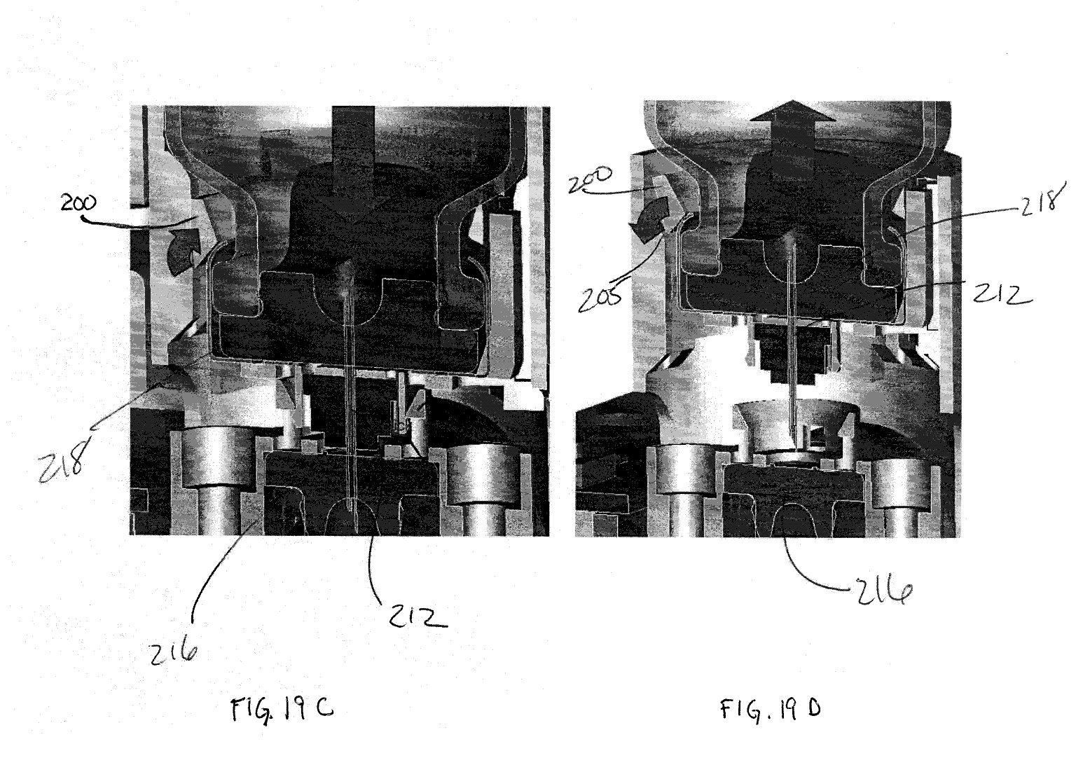

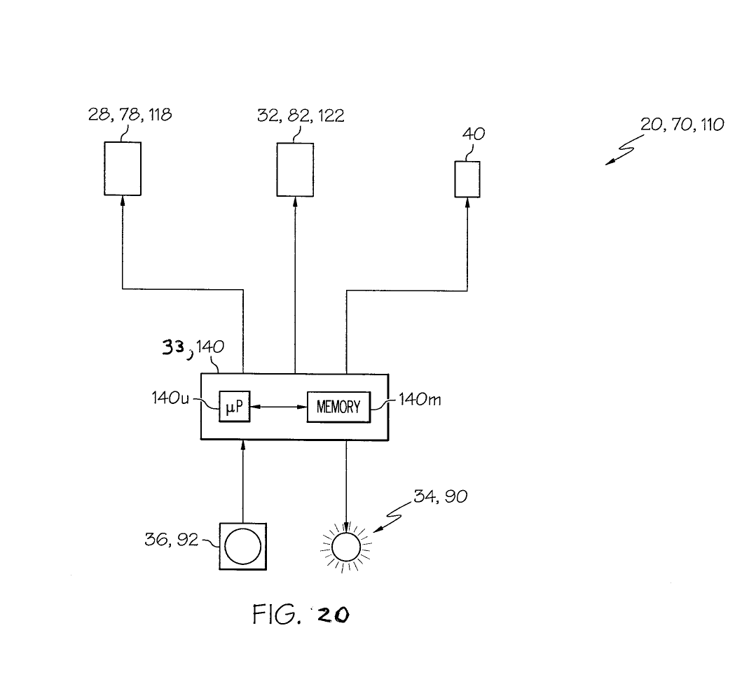

FIGS. 19A-19D show an alternative connection system that can also be incorporated into the disclosed device. In this embodiment, collet 200 that is part of needle shuttle 208 is used to control the sequence of inserting and removing the vial 202 and also making and breaking the fluidic connection between the vial and the cartridge. In its rest position shown in FIG. 19A, collet 200 is displaced outwards and contacts a feature 205 on the inner surface of needle shuttle housing 204 that prevents downward movement of needle shuttle 208. In FIG. 19 B after the vial 202 is inserted into needle shuttle housing 204 and past collet 200, the collet is forced inwards away from feature 205 and allows downward movement of needle shuttle 208. At the same time, needle 212 pierces a septum of the vial 218. As shown in FIG. 19C, once vial 202 moves downward and contacts needle shuttle 208 on surface 209 it then pushes the entire needle shuttle 208 downward, and a second end of needle 212 penetrates septum 216 thereby establishing a fluidic connection between the vial 202 and a cartridge (not shown). Collet 200 is now encircling the head of vial 202. Thus, on removal of the vial, collet 200 ensures that needle shuttle 208 is pulled upwards and needle 212 is removed from septum 216. This process is further shown in FIG. 19 D, and at the end of the removal process, collet 200 returns to its rest position on feature 205, allowing removal of vial 202 and thereby removal of needle 212 from vial septum 218. In this process, the fluidic connection between the needle and the cartridge is broken before the connection between the vial and the needle is broken, thereby reducing the likelihood of operator error.

FIG. 20 depicts an electrical schematic of a drug delivery device 20, 70, 110 according to one or more embodiments shown and described herein. The device 20, 70, 110 may be capable of automatically reconstituting a drug such as a lyophilized drug and automatically delivering the reconstituted drug to the user. The device 20, 70, 110 may comprise, inter alia, a cartridge drive 28, 78, 118, an injection needle drive 32, 82, 122, a cartridge needle drive 40, an annunciator 34, 90, a user input 36, 92, and a controller 33, 140. The cartridge drive 28, 78, 118 may be mechanically coupled to the cartridge and may be able to change pressure of fluid in the cartridge to transfer fluid into or out of the cartridge, as described herein. The cartridge drive 28, 78, 118 may comprise an electric motor as the actuator. It is contemplated that other types of actuators may be used as well such as, for example, piezoelectric actuators and electro-active polymers. The controller 33, 140 may be electrically coupled to the cartridge drive 28, 78, 118 such that the controller 33, 140 controls transfer of fluid into or out of the cartridge by activating the cartridge drive 28, 78, 118.

If the actuator is an electric motor, the controller 33, 140 controls transfer of fluid into or out of the cartridge by activating the electric motor. In this embodiment, the electric motor may comprise a DC electric motor which rotates in one direction when a positive electrical current is applied to it, and which rotates in the opposite direction when a negative electrical current is applied to it. In this manner, the controller 33, 140 may control the direction of rotation of the motor which correspondingly controls whether the cartridge drive 28, 78, 118 transfers fluid into or out of the cartridge. The controller 33, 140 may further comprise a power circuit (not shown) for the motor in order to step up the voltage and/or current to a suitable level for driving the motor.

The cartridge drive 28, 78, 118 may further comprise one or more sensors (not shown) in order to provide feedback to the controller 33, 140 regarding the state of the cartridge drive 28, 78, 118. For example, a position sensor may be disposed on the plunger (e.g., the plunger disposed in the cartridge) in order to allow the controller 33, 140 to ascertain the position of the plunger. This may allow the controller 33, 140 to control accurately the amount of fluid transferred into or out of the cartridge as well as the rate of the transfer. As an alternative, the cartridge drive 28, 78, 118 may have one or more proximity sensors to detect when the plunger is fully extended or fully retracted. In this embodiment, the controller 33, 140 may activate the cartridge drive 28, 78, 118 to transfer fluid into or out of the cartridge until the one or more sensors indicate that the plunger is fully extended (e.g., for transferring fluid out of the cartridge) or fully retracted (e.g., for transferring fluid into the cartridge), at which time the controller 33, 140 deactivates the cartridge drive 28, 78, 118. In summary, there are numerous ways for the controller 33, 140 to activate the cartridge drive 28, 78, 118 and control the transfer of fluid into or out of the cartridge.

Referring still to FIG. 20, the injection needle drive 32, 82, 122 may be mechanically coupled to the injection needle and may be capable of inserting the injection needle into the body of the user when the injection needle drive 32, 82, 122 is activated. The injection needle drive 32, 82, 122 may comprise a solenoid and a spring, as previously described herein. The spring may act to force the injection needle to remain inside the housing of the device 20, 70, 110 when the injection needle drive 32, 82, 122 is deactivated. Conversely, activation of the solenoid causes the injection needle to overcome the force of the spring such that the injection needle is inserted automatically into the body of the user. The controller 33, 140 may be electrically coupled to the injection needle drive 32, 82, 122 such that the controller 33, 140 controls whether the injection needle drive 32, 82, 122 is activated or deactivated. The controller 33, 140 may activate the injection needle drive 32, 82, 122 by passing a suitable current through the solenoid. It is contemplated that other types of actuators may be used as well such as, for example, piezoelectric actuators, shape memory alloy drives and electro-active polymers.

The injection needle drive 32, 82, 122 may further comprise one or more sensors (not shown) in order to provide feedback to the controller 33, 140 regarding the state of the injection needle drive 32, 82, 122. For example, a position sensor may be disposed on the injection needle in order to allow the controller 33, 140 to determine the position of the injection needle. As an alternative, the injection needle may have one or more proximity sensors to detect whether the injection needle is fully retracted in the housing or fully inserted into the body of the user. This may allow the controller 33, 140 to determine whether there are any faults regarding the activation of the injection needle drive 32, 82, 122. For example, the use of a proximity sensor on the injection needle may permit the controller 33, 140 to ascertain whether the injection needle was fully inserted into the body of the user (i.e., after the injection needle drive 32, 82, 122 was activated).

Referring still to FIG. 20, the cartridge needle drive 40 may be mechanically coupled to the cartridge needle and may be capable of inserting the cartridge needle into the cartridge when the cartridge needle drive 40 is activated. Not all embodiments of the drug delivery device 20, 70, 110 comprise a cartridge needle drive 40. The cartridge needle drive 40 may comprise a solenoid and a spring, as previously described herein. The spring may act to force the cartridge needle to remain fluidly decoupled from the cartridge when the cartridge needle drive 40 is deactivated. Conversely, activation of the solenoid causes the cartridge needle to overcome the force of the spring such that the injection needle is inserted automatically into the cartridge. The controller 33, 140 may be electrically coupled to the injection needle drive 32, 82, 122 such that the controller 33, 140 controls whether the cartridge needle drive 40 is activated or deactivated. The controller 33, 140 may activate the cartridge needle drive 40 by passing a suitable current through the solenoid. It is contemplated that other types of actuators may be used as well such as, for example, piezoelectric actuators and electro-active polymers.

The cartridge needle drive 40 may further comprise one or more sensors (not shown) in order to provide feedback to the controller 33, 140 regarding the state of the cartridge needle drive 40. For example, a position sensor may be disposed on the cartridge needle in order to allow the controller 33, 140 to determine the position of the cartridge needle. As an alternative, the cartridge needle may have one or more proximity sensors to detect whether the cartridge needle is fully retracted or fully inserted into the cartridge. This may allow the controller 33, 140 to determine whether there are any faults regarding the activation of the cartridge needle drive 40. For example, the use of a proximity sensor on the cartridge needle may permit the controller 33, 140 to ascertain whether the cartridge needle was fully inserted into the cartridge (i.e., after the cartridge needle drive 40 was activated).

The user input 36, 92 may comprise a pushbutton, a switch, or other suitable device. The user input 36, 92 may be electrically coupled to the controller 33, 140 such that the controller 33, 140 is able to determine whether the user is activating the user input 36, 92. In one embodiment, the user may insert the vial into the connector and activate (e.g., press) the user input 36, 92 to inform the controller 33, 140 that the reconstitution process is ready to begin. The annunciator 34, 90 may comprise a light, a light emitting diode (LED), or other suitable device. The annunciator 34, 90 may be electrically coupled to the controller 33, 140 such that the controller 33, 140 controls the activation of the annunciator 34, 90. For example, if the annunciator 34, 90 is a light, the controller 33, 140 controls whether the annunciator 34, 90 is activated (e.g., illuminated) or deactivated (e.g., extinguished). The annunciator 34, 90 may comprise other types of devices such as, for example, acoustic devices, vibratory devices, or combinations thereof. In the embodiment shown herein, the user input 36, 92 is a pushbutton, and the annunciator 34, 90 is an LED which surrounds the pushbutton as an annular ring. The annunciator 34, 90 may indicate to the user the status of the drug delivery device 20, 70, 110. For example, the annunciator 34, 90 may flash when the drug has been reconstituted and the vial is ready to be removed from the device. The annunciator 34, 90 may also indicate other status information such as, for example, whether an error occurred during the reconstitution process, whether the battery is low, etc.

Referring still to FIG. 20, the controller 33, 140 may comprise a microcontroller 33, 140u and a memory 140m. The microcontroller 33, 140u may be a 4-bit, 8-bit, 16-bit, or any other suitable device. For example, the microcontroller 33, 140u may be an 8-bit device available from Microchip Technologies located in Chandler, Ariz. It is contemplated that other microcontrollers, from both Microchip Technologies and other manufacturers, may be used as well.

The microcontroller 33, 140u may be electrically coupled to the memory 140m such that the microcontroller 33, 140u is capable of executing computer-readable and computer-executable instructions stored in the memory 140m. In one embodiment, the microcontroller 33, 140u and the memory 140m reside on the same monolithic device. The computer-readable and computerexecutable instructions stored in the memory 140m may embody one or more of the methods described herein to automatically reconstitute a drug such as a lyophilized drug and automatically delivering the reconstituted drug to the user.

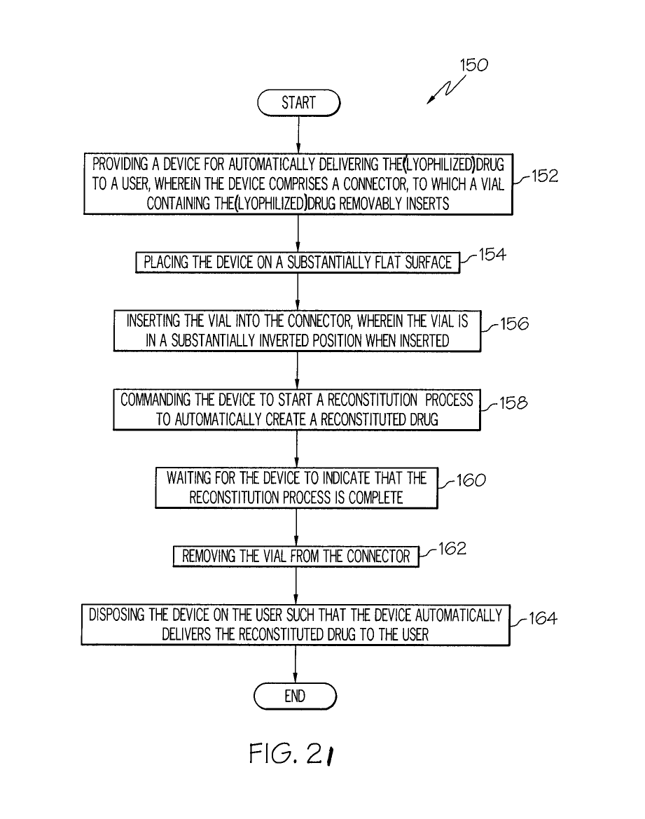

FIG. 21 depicts a flow diagram of a method 150 for automatically reconstituting a drug such as, for example, a lyophilized drug and automatically delivering the reconstituted drug to a patient, such as the user or a different person, according to one embodiment. The method 150 may comprise a number of steps which may be performed in any suitable order. At step 152, a device, such as device 10, is provided for automatically delivering the drug to a user, wherein the device comprises a connector, to which a vial containing the drug removably inserts. At step 154, the user places the device on a substantially flat surface. At step 156, the user inserts the vial into the connector, wherein the vial is in a substantially inverted position when inserted. At step 158, the user commands the device to start a reconstitution process to create automatically a reconstituted drug. At step 160, the user waits for the device to indicate that the reconstitution process is complete. At step 162, the user removes the vial from the connector. In addition, at step 164, the user disposes the device on the user such that the device automatically delivers the reconstituted drug to the user.

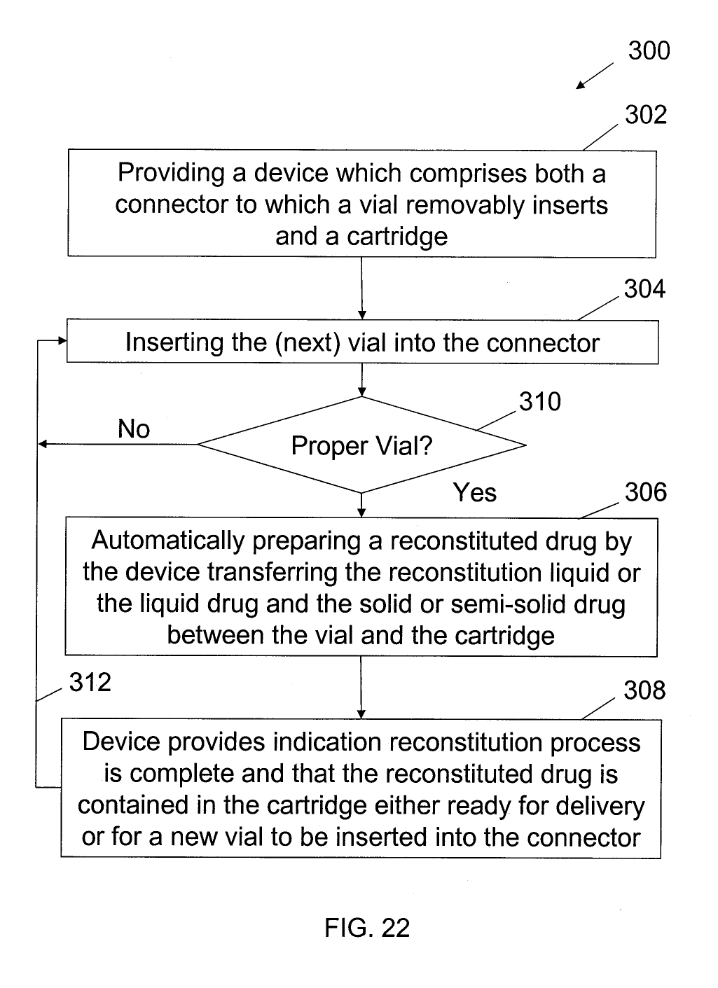

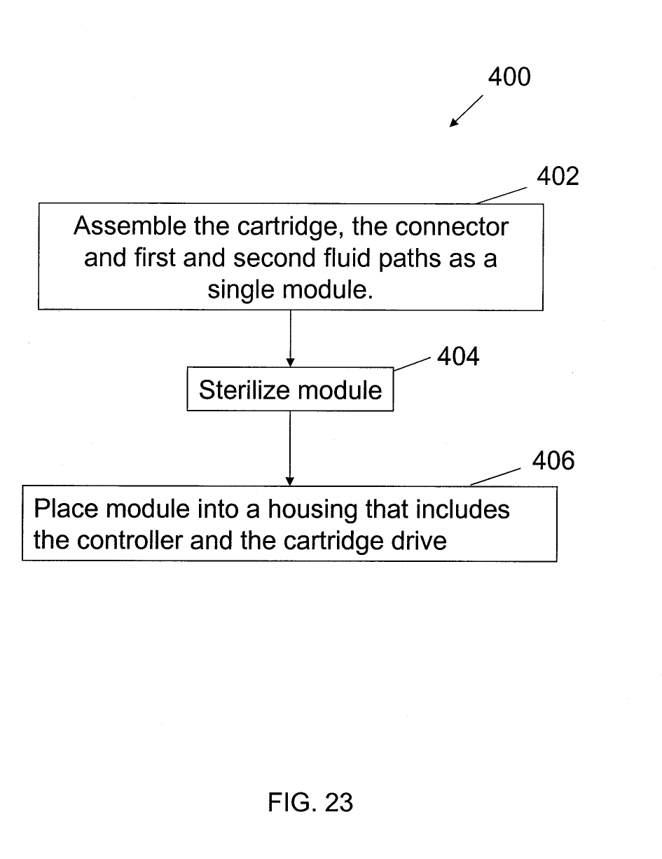

FIG. 22 depicts another embodiment of another method 300 that can be carried out with the device 10, such as a method for automatically reconstituting a drug. The method 300 may comprise a number of steps which may be performed in any suitable order. In step 302, the method comprises providing a device, such as device 10, which comprises both a connector to which a vial removably inserts and a cartridge. It is to be appreciated that the cartridge can initially be empty, contain a reconstitution liquid or a liquid drug, or contain a solid or semi-solid drug. In step 304, the method comprises inserting the vial into the connector, wherein if the cartridge is initially empty or contains the solid or semi-solid drug the vial contains either the liquid drug or the reconstitution fluid, and wherein if the cartridge initially contains the reconstitution fluid or the liquid drug the vial contains the solid or semi-solid drug. In step 306, the device automatically prepares a reconstituted drug by transferring the reconstitution liquid or the liquid drug and the solid or semi-solid drug between the vial and the cartridge. At step 308, once the reconstitution process is complete the device automatically indicates that the reconstitution process is complete and that the reconstituted drug is contained in the cartridge either ready for delivery or for a new vial to be inserted into the connector.

In still other embodiments, after the vial is inserted into the connector, the reconstitution process starts after receiving user input to start i.e., the user commands the device to start a reconstitution process to create automatically the reconstituted drug. In another embodiment, when the vial is inserted into the connector, the device starts automatically the reconstitution process. In still another embodiment, the device will automatically starts the reconstitution process after the device confirms in step 310 that the proper vial has been inserted into the connector. For example, in such an embodiment, the device confirms the proper vial has been inserted into the connector via electronic identification, such as by RFID, barcode, magnetic strip, and the likes. In other embodiments, the method 300 includes indicating for the removal of the vial, such as via the annunciator, and such as after the reconstitution process or at an intermediate point in which another vial should be connected to the connector in order to continue the reconstitution process. If stopping the reconstitution process at an intermediate point, then the method 300 comprises waiting for a vial change in step 312. After each vial change, the method 300 the resumes automatically the reconstitution process after the device either receives user input of the vial change or confirms automatically that the proper vial has been inserted into the connector, such as via electronic identification as mentioned above.

In another embodiment, the method 300 further comprises transferring the reconstitution liquid or the liquid drug and the solid or semi-solid drug between the vial and the cartridge one or more times to prepare the reconstituted drug. In still other embodiments, the method 300 further comprises the user waits for the device to indicate that the reconstitution process is complete, the user optionally removing the vial from the connector, and then disposing the device on a patient such that the device automatically delivers the reconstituted drug to the patient, such as previously described above in method 150 (FIG. 21). It is to be appreciated that in some embodiments the device when disposed on the patient will need to be orientated such that the vial is in an inverted position. By inverted position, it is meant any position in which the bottom surface of vial is higher than its top surface, e.g., from a substantially inverted position where the bottom surface is directly above the top surface to a titled position in which the bottom surface is slightly higher vertically than the top surface.

It should now be understood that the devices and methods described herein may permit a user to automatically reconstitute a drug and automatically deliver the reconstituted drug to the user. Automation increases safety because it can reduce exposure of healthcare workers to potentially toxic substances in any case, and further permits reconstitution to be performed in a safe environment such as under a laminar flow hood. An advantage of certain embodiments is that due to the automated reconstitution process the liquid transfer between cartridge and vial takes place under flow conditions avoiding turbulences during the transfer processes. Drug specific reconstitution times can be predefined so that administering can start at earliest after the time obliged for reconstitution. Still another advantage of certain embodiments is that after completing the reconstitution process, the cartridge is filled entirely with the reconstituted drug (which is not the case for in line reconstitution syringes which include a certain amount of air after reconstitution and therefore need to be expelled by a nurse before administering). Because the vial during the reconstitution process can be located higher than the cartridge, the vial can work as bubble trap. Furthermore, either the whole of the device or only those parts in contact with the drug can be easily sterilized because the device can be provided separately from the drug (either with or without a reconstitution fluid contained therein). For example, the cartridge and fluid path including body and septum needles can be provided in a sealed container that is then sterilized, e.g. by methods such as gamma sterilization, e-beam sterilization, autoclaving, and the likes, prior to being assembled into the rest of the drug delivery device. The cartridge can be filled with a reconstitution liquid, a drug or be empty before the sterilization process takes place. In still another example, the cartridge containing the liquid for reconstituting the drug, the connector, and the second fluid path can be manufactured as a sterilized module which is then assembled into the rest of the drug deliver device.

For example, a method 400 of assembling the device 10 is a follows. In step 402, the cartridge, the connector and first and second fluid paths by are assembled as a single module. In step 404, the module is sterilized, and placed into a housing that includes the controller and the cartridge drive in step 406 which assembles the device. The assembly could be done at a factory for a completely disposable device, or the housing could be a reusable housing which is supplied to a user, such as a doctor, a nurse or a patient. In such an embodiment, the module is provided in a sterilized and sealed container which maintains the sterility for the shelf-life of the module. The module is then also supplied to the user, which removes the module from the container and assembles the complete unit (reusable housing plus module) at the point of care.

While particular embodiments and aspects of the present invention have been illustrated and described herein, various other changes and modifications may be made without departing from the spirit and scope of the invention. Moreover, although various inventive aspects have been described herein, such aspects need not be utilized in combination. It is therefore intended that the appended claims cover all such changes and modifications that are within the scope of this invention.

1. A device which automatically reconstitutes and delivers a drug to a user, comprising:
a controller;
a cartridge;
a connector to which a vial removably inserts, wherein inserting the vial into the connector causes the vial to be fluidly coupled to the cartridge through the connector along a first fluid path;
a reversible cartridge drive both electrically coupled to the controller such that the controller controls activation of the cartridge drive and mechanically coupled to the cartridge such that activation of the cartridge drive causes fluid to be either transferred into or out of the cartridge; and
a second fluid path which is formed following reconstitution to enable a reconstituted drug in the cartridge to be delivered to the user.

2. The device of claim 1, wherein the cartridge contains a liquid for reconstituting the drug, and wherein the cartridge containing the liquid, the connector, and the second fluid path are manufactured as a sterilized module.

3. The device of claim 1, wherein the controller waits for the user to indicate via a user input that the vial is removed from the connector and that the device is physically coupled to the user before forming the second fluid path.

4. The device of claim 1, wherein the second fluid path is formed by the controller automatically fluidly coupling a cartridge needle of the device to the cartridge.

5. The device of claim 1, wherein the second fluid path comprises an injection needle, and wherein the controller automatically fluidly couples the injection needle to the user, and automatically delivers the reconstituted drug from the cartridge, through the injection needle, and to the user, by activating the cartridge drive in a first manner.

6. The device of claim 5, wherein the cartridge drive can be activated in a second manner by the controller, and wherein activating the cartridge drive in the first manner causes the fluid to be transferred out of the cartridge by increasing pressure on the fluid in the cartridge, and activating the cartridge drive in the second manner causes the fluid to be transferred into the cartridge by decreasing pressure on the fluid in the cartridge.

7. The device of claim 1, wherein the first fluid path comprises a cartridge needle which is insertable into the cartridge.

8. The device of claim 1, further comprising a cartridge needle drive and the first fluid path comprises a cartridge needle, wherein the cartridge needle drive inserts the cartridge needle into the cartridge.

9. The device of claim 8, wherein the cartridge is moved in a first direction by the cartridge drive to insert the cartridge needle into the cartridge.

10. The device of claim 1, wherein the second fluid path comprises an injection needle.

11. The device of claim 10, further comprising an injection needle drive both electrically coupled to the controller such that the controller controls activation of the injection needle drive and mechanically coupled to the injection needle such that, when the device is physically coupled to the user and the injection needle drive is activated by the controller, the injection needle drive inserts the injection needle into the user to fluidly couple the injection needle to the user.

12. The device of claim 1, further comprising an annunciator electrically coupled to the controller and in which the controller automatically activates the annunciator to indicate that the reconstituted drug is disposed in the cartridge and the vial is ready to be removed.

13. The device of claim 12, wherein the annunciator comprises a light visible to the user, and the controller activates the light by illuminating the light.

14. The device of claim 1, further comprising a user input, wherein the user input is automatically activated by the controller when the vial is inserted into the connector.

15. The device of claim 14, wherein the user input comprises a pushbutton.

16. The device of claim 1, wherein the device agitates the reconstituted drug.

17. The device of claim 16, wherein the device agitates the reconstituted drug by activating the cartridge drive one or more times in a first manner and then in a second manner.

18. The device of claim 17, wherein a stroke of the cartridge drive during agitation is less than the volume of the reconstituted drug.

19. The device of claim 1, wherein the controller transfers the reconstituted drug out of the vial, through the connector, and into the cartridge, by activating the cartridge drive in a first manner, and automatically transfers the reconstituted drug out of the vial, through the connector, and into the cartridge by activating the cartridge drive in a second manner.

20. The device of claim 1, wherein the liquid comprises water for injection.

21. The device of claim 1, wherein the device is configured such that, when the vial is inserted into the connector, the vial must be substantially inverted.

22. The device of claim 1, further comprising a sensor electrically coupled to the controller and mechanically coupled to the device such that:
the sensor senses whether the device is disposed on a surface; and
if the device is not disposed on the surface, the controller automatically inhibits transferring the liquid out of the cartridge and into the vial containing the drug.

23. The device of claim 8, wherein the cartridge needle drive is both electrically coupled to the controller such that the controller controls activation of the cartridge needle drive and mechanically coupled to the cartridge needle such that activation of the cartridge needle drive inserts the cartridge needle into the cartridge to fluidly couple the cartridge to the injection needle.

24. The device of claim 23, wherein the controller automatically activates the cartridge needle drive after receiving indication via a user input that the vial is removed from the connector and that the device is physically coupled to the user.

25. The device of claim 23, wherein the controller waits for the injection needle to be fluidly coupled to the cartridge by waiting for the cartridge needle drive to activate completely.

26. The device of claim 1, wherein, when the vial is inserted in the connector, a vial conduit disposed in the vial fluidly couples the vial to the cartridge through the vial conduit.

27. The device of claim 26, wherein the cartridge comprises a septum, and, when the vial is inserted into the connector, the vial conduit punctures the septum and fluidly couples the vial to the cartridge through the vial conduit.

28. The device of claim 1, wherein the vial comprises a shuttle needle, a guide, and a vial septum, and the cartridge comprises a collet having a cartridge septum, wherein:
the shuttle needle is mechanically coupled to the guide such that the guide permits the shuttle need to move along a longitudinal axis of the vial; and
when the vial is inserted into the connector, the guide contacts the collet and causes the shuttle needle to pierce both the vial septum and the cartridge septum.

29. The device of claim 1, wherein:
the connector is rotatable about a longitudinal axis of the connector;
when the vial is inserted into the connector, the vial and the connector are physically coupled such that the connector and the vial rotate in unison around the longitudinal axis;
when the vial is inserted into the connector, rotation of the vial in a first rotational direction by a locked angle amount causes the vial to be locked in the connector; and
when the vial is inserted into the connector and rotated so as to be physically locked in the connector, the vial must be rotated in the first rotational direction or in a second rotational direction by the locked angle amount in order to be removed.

30. The device of claim 29, further comprising a cartridge Needle and a cartridge plenum, wherein:
the cartridge needle is fluidly coupled to the injection needle;
the cartridge plenum is fluidly coupled to the cartridge and mechanically coupled to the connector such that, when the vial is rotated in the first rotational direction or the second rotational direction by the locked angle amount in order to be removed, such rotation of the connector causes the cartridge plenum to be fluidly coupled to the cartridge needle; and
the controller waits for the injection needle to be fluidly coupled to the cartridge by waiting for the user to indicate via a user input that the vial is removed from the connector and that the device is physically coupled to the user.

31. The device of claim 29, wherein the locked angle amount is about 45 degrees.

32. The device of claim 29, the connector provides a vial conduit which fluidly couples the vial to the cartridge when the vial is inserted into the connector.

33. The device of claim 1, further comprising an arcuate rail and an injection needle conduit, wherein:
the arcuate rail is fixedly coupled to the device and slidingly coupled to the vial when the vial is inserted into the connector;
the connector is configured to rotate about a longitudinal axis of the cartridge such that the connector is in a first orientation when the vial is inserted into the connector;
the injection needle conduit is fluidly coupled to the injection needle and mechanically coupled to the connector such that when the connector is at the first orientation, the injection needle conduit is fluidly uncoupled from the cartridge;
the user rotates the connector, and correspondingly the vial, to a second orientation when the user removes the vial from the connector, wherein rotating the connector from the first orientation to the second orientation causes:
the arcuate rail to slidingly engage the vial so as to push the vial out of the connector, and
the injection needle conduit to be fluidly coupled to the cartridge.

34. The device of claim 33, wherein the first orientation of the connector causes the vial to be inserted into the connector when the vial is in a substantially inverted position, and wherein the second orientation of the connector causes the vial to be removed from the connector when the vial is in a substantially horizontal position.

35. The device of claim 33, wherein the connector provides a vial conduit which fluidly couples the vial to the cartridge when the vial is inserted into the connector.

36. The device of claim 1, wherein the connector comprises a needle shuttle.

37. The device of claim 36 wherein a mechanism ensures that the needle is inserted into the vial before the needle shuttle moves to insert the needle into the cartridge.

38. The device of claim 36 wherein a mechanism ensures that the needle shuttle is removed from the cartridge and locked in place before the vial is removed from the needle shuttle.

39. A method for automatically reconstituting and delivering a drug to a user which comprises utilizing the device of claim 1.

40. A method for automatically reconstituting a drug, comprising:
providing a device which comprises a connector to which a vial removably inserts and also comprises a cartridge, wherein the cartridge can initially be empty, contain a reconstitution liquid or a liquid drug, or contain a solid or semi-solid drug; and
inserting the vial into the connector, wherein if the cartridge is initially empty or contains the solid or semi-solid drug, the vial contains either the liquid drug or the reconstitution fluid, and wherein if the cartridge initially contains the reconstitution fluid or the liquid drug the vial contains the solid or semi-solid drug,
wherein the device automatically prepares a reconstituted drug by transferring the reconstitution liquid or the liquid drug and the solid or semi-solid drug between the vial and the cartridge, and
wherein once the reconstitution process is complete the device automatically indicates that the reconstitution process is complete and that the reconstituted drug is contained in the cartridge either ready for delivery or for a new vial to be inserted into the connector.

41. The method of claim 40, further comprises transferring the reconstitution liquid or the liquid drug and the solid or semi-solid drug between the vial and the cartridge one or more times to prepare the reconstituted drug.

42. The method of claim 40, further comprises disposing the device on a patient such that the device automatically delivers the reconstituted drug to the patient.

43. The method of claim 40, wherein after said vial is inserted into the connector, the reconstitution process starts after receiving user input to start.

44. The method of claim 40, wherein when said vial is inserted into the connector, the device starts automatically the reconstitution process.

45. The method of claim 44, wherein the device automatically starts the reconstitution process after the device confirms that the proper vial has been inserted into the connector.

46. A method for automatically reconstituting a drug, the method comprising:
providing a device for automatically delivering the drug to a user, wherein the device comprises a connector, to which a vial containing the drug removably inserts;
placing the device on a substantially flat surface;
inserting the vial into the connector, wherein the vial is in a substantially inverted position when inserted;
commanding the device to start a reconstitution process to automatically create a reconstituted drug;
waiting for the device to indicate that the reconstitution process is complete;
removing the vial from the connector; and
disposing the device on the user such that the device automatically delivers the reconstituted drug to the user.

47. The method of claim 46, wherein inserting the vial into the connector comprises pressing the vial into the connector and rotating the vial in a first rotational direction by a locked angle amount about a longitudinal axis of the vial.

48. The method of claim 47, wherein removing the vial from the connector comprises rotating the vial in the first rotational direction or in a second rotational direction by the locked angle amount around the longitudinal axis of the vial and pulling the vial from the connector.

49. A method of assembling the device of claim 1 by assembling the cartridge, connector and first and second fluid paths as a single module; sterilizing the module; and placing the module into a housing including the controller and cartridge drive to assemble the device.
